SEQUENCE LISTING (37 sequences)

SEQ ID NO: 1 - DNA, 24 bp, Artificial Sequence
  A novel FISSR-PCR primers for genotyping eukaryotes
gatgctgatacacacacacacaca

SEQ ID NO: 2 - DNA, 24 bp, Artificial Sequence
  A novel FISSR-PCR primer for genotyping eukaryotes
gcacatgcagtgtgtgtgtgtgtg

SEQ ID NO: 3 - DNA, 24 bp, Artificial Sequence
  Novel FISSR-PCR primer for genotyping eukaryotes
catgcacattgtgtgtgtgtgtgt

SEQ ID NO: 4 - DNA, 24 bp, Artificial Sequence
  A novel FISSR-PCR primer for genotyping eukaryotes
gctagtgctcacacacacacacac

SEQ ID NO: 5 - DNA, 23 bp, Artificial Sequence
  A novel FISSR-PCR primer for genotyping eukaryotes
cgtatgtgtgtgtgtgtgtgtgt

SEQ ID NO: 6 - DNA, 22 bp, Artificial Sequence
  A novel FISSR-PCR primer for genotyping eukaryotes
tgtaatgagagagagagagaga

SEQ ID NO: 7 - DNA, 22 bp, Artificial Sequence
  A novel FISSR-PCR primer for genotyping eukaryotes
gacgatacgagagagagagaga

SEQ ID NO: 8 - DNA, 18 bp, Artificial Sequence
  A novel FISSR-PCR primer for genotyping eukaryotes
cccgggattattattatt

SEQ ID NO: 9 - DNA, 17 bp, Artificial Sequence
  A novel FISSR-PCR primer for genotyping eukaryotes
aaatacagcagcagcag

SEQ ID NO: 10 - DNA, 17 bp, Artificial Sequence
  A novel FISSR-PCR primer for genotyping eukaryotes
gtgctaataataataat

SEQ ID NO: 11 - DNA, 17 bp, Artificial Sequence
  A novel FISSR-PCR primer for genotyping eukaryotes
aatttattattattatt

SEQ ID NO: 12 - DNA, 17 bp, Artificial Sequence
  A novel FISSR-PCR primer for genotyping eukaryotes
gagtcattattattatt

SEQ ID NO: 13 - DNA, 17 bp, Artificial Sequence
  A novel FISSR-PCR primer for genotyping eukaryotes
agcgaattattattatt

SEQ ID NO: 14 - DNA, 17 bp, Artificial Sequence
  A novel FISSR-PCR primer for genotyping eukaryotes
taaaaaataataataat

SEQ ID NO: 15 - DNA, 17 bp, Artificial Sequence
  A novel FISSR-PCR primer for genotyping eukaryotes
acaaaaataataataat

SEQ ID NO: 16 - DNA, 17 bp, Artificial Sequence
  A novel FISSR-PCR primer for genotyping eukaryotes
agtgaattattattatt

SEQ ID NO: 17 - DNA, 17 bp, Artificial Sequence
  A novel FISSR-PCR primer for genotyping eukaryotes
gtgatattattattatt

SEQ ID NO: 18 - DNA, 17 bp, Artificial Sequence
  A novel FISSR-PCR primer for genotyping eukaryotes
tgagcgccgccgccgcc

SEQ ID NO: 19 - DNA, 27 bp, Artificial Sequence
  A novel FISSR-PCR primer for genotyping eukaryotes
tcgataccaccaccaccaccaccacca

SEQ ID NO: 20 - DNA, 23 bp, Artificial Sequence
  A novel FISSR-PCR primer for genotyping eukaryotes
atagagctgctgctgctgctgct

SEQ ID NO: 21 - DNA, 18 bp, Artificial Sequence
  A novel FISSR-PCR primer for genotyping eukaryotes
aatcgaaagaagaagaag

SEQ ID NO: 22 - DNA, 18 bp, Artificial Sequence
  A novel FISSR-PCR primer for genotyping eukaryotes
cataataagaagaagaag

SEQ ID NO: 23 - DNA, 22 bp, Artificial Sequence
  A novel FISSR-PCR primer for genotyping eukaryotes
atcgaataataataataataat

SEQ ID NO: 24 - DNA, 15 bp, Artificial Sequence
  A novel FISSR-PCR primer for genotyping eukaryotes
gcatatgagatgatg

SEQ ID NO: 25 - DNA, 19 bp, Artificial Sequence
  A novel FISSR-PCR primer for genotyping eukaryotes
atggacagacagacagaca

SEQ ID NO: 26 - DNA, 20 bp, Artificial Sequence
  A novel FISSR-PCR primer for genotyping eukaryotes
gtgtgtgtgtgtgtgtatcc

SEQ ID NO: 27 - DNA, 19 bp, Artificial Sequence
  A novel FISSR-PCR primer for genotyping eukaryotes
gagagagagagagagacgg

SEQ ID NO: 28 - DNA, 17 bp, Artificial Sequence
  A novel FISSR-PCR primer for genotyping eukaryotes
aagaagaagaagaacta

SEQ ID NO: 29 - DNA, 17 bp, Artificial Sequence
  A novel FISSR-PCR primer for genotyping eukaryotes
aagaagaagaagtacga

SEQ ID NO: 30 - DNA, 17 bp, Artificial Sequence
  A novel FISSR-PCR primer for genotyping eukaryotes
cttcttcttcttatgct

SEQ ID NO: 31 - DNA, 17 bp, Artificial Sequence
  A novel FISSR-PCR primer for genotyping eukaryotes
ggcggcggcggcgctaa

SEQ ID NO: 32 - DNA, 16 bp, Artificial Sequence
  A novel FISSR-PCR primer for genotyping eukaryotes
atgatgatgatggact

SEQ ID NO: 33 - DNA, 15 bp, Artificial Sequence
  A novel FISSR-PCR primer for genotyping eukaryotes
aaacaaacaaacatc

SEQ ID NO: 34 - DNA, 23 bp, Artificial Sequence
  A novel FISSR-PCR primer for genotyping eukaryotes
cacacacacacacaatgcacagc

SEQ ID NO: 35 - DNA, 19 bp, Artificial Sequence
  A novel FISSR-PCR primer for genotyping eukaryotes
gagagagagagagaactat

SEQ ID NO: 36 - DNA, 18 bp, Artificial Sequence
  A novel FISSR-PCR primer for genotyping eukaryotes
gccgccgccgccgcactc

SEQ ID NO: 37 - DNA, 14 bp, Artificial Sequence
  A novel FISSR-PCR primer for genotyping eukaryotes
aacaacaacaacgt

(19) United States
(12) Patent Application Publication
(10) Pub. No.: US 2003/0194730 A1
(43) Pub. Date: Oct. 16, 2003

(54) Novel FISSR-PCR primers and methods of identifying genotyping diverse genomes of plant and animal systems including rice varieties, a kit thereof

(75) Inventor: Javare Gowda Nagaraju, Hyderabad (IN)

Correspondence Address: SUGHRUE MION, PLLC, 2100 PENNSYLVANIA AVENUE, N.W., WASHINGTON, DC, 20037, US

(73) Assignee: CENTRE FOR DNA FINGERPRINTING AND DIAGNOSTICS (CDFD)

(21) Appl. No.: 10/357,488

(22) Filed: Feb. 4, 2003

(30) Foreign Application Priority Data
Apr. 8, 2002 (IN) 260/MAS/2002

(51) Int. Cl. C12Q 1/68; C12P 19/34

(52) U.S. Cl. 435/6; 435/91.2

(57) ABSTRACT

The present invention relates to set of inter-simple sequence repeats (ISSR)-PCR primers of SEQ ID Nos. 1 to 37 for genotyping eukaryotes and a method of genotyping diverse genomes of plant and animal systems using FISSR-PCR primers and SSR markers; more particularly, a FISSR and SSR method of distinguishing Basmati rice varieties from Non-Basmati (NB) rice varieties, and Traditional Basmati (TB) rice varieties from Evolved Basmati (NB) rice varieties, and also, a method for determining adulteration of Basmati rice with other rice varieties and a kit thereof.

### FIELD OF THE PRESENT INVENTION

The present invention relates to a set of inter-simple sequence repeats (ISSR)-PCR primers of SEQ ID Nos. 1 to 37 for genotyping eukaryotes and a method of genotyping diverse genomes of plant and animal systems using FISSR-PCR primers and SSR markers; more particularly, a FISSR and SSR method of distinguishing Basmati rice varieties from Non-Basmati (NB) rice varieties, and Traditional Basmati (TB) rice varieties from Evolved Basmati (NB) rice varieties, and also, a method for determining adulteration of Basmati rice with other rice varieties and a kit thereof.

### BACKGROUND AND PRIOR ART REFERENCE OF THE PRESENT INVENTION

Rice is the staple food for more than half of the world's population. Its rich genetic diversity in the form of thousands of land races and progenitor species besides its economic significance have aroused unending interest among scientists for several decades. In the evolution of rice and its genetic differentiation into distinct varietal groups, consumer quality preferences have played a significant role besides agro-ecological factors. One such varietal group comprising of aromatic pulao/biryani rice of Indian sub-continent known as ‘Basmati’ is the highly priced rice in the domestic as well as international markets. Originated in the foothills of the Himalayas Basmati rice is characterized by extra long slender grain, pleasant and distinct aroma and soft and fluffy texture of cooked rice. These unique features of Basmati said to be the culmination of centuries of selection and cultivation by farmers, are well preserved and maintained in their purest form in the traditional Basmati (TB) varieties.

The historical and archeological findings infer that the varieties with such unique morphological and quality attributes are not present in traditional rice growing areas anywhere in the world¹. A number of undesirable traits of Basmati such as tall stature, low yield, sensitivity to photoperiod and poor response to fertilizer application prompted breeders to develop ‘elite’ Basmati varieties by making use of the high yielding semi-dwarf non-Basmati (NB) rice varieties. Such ‘elite’ evolved lines of Basmati (EB), however, fall short of the quality features of traditional varieties. Difficulty in recovering desirable recombinants from crosses involving NB and TB varieties, and reversion often to parental types in the backcross generations, suggest that probably indica and Basmati types are phylogenetically divergent². A study on Asian rice varieties using isozyme markers clustered Basmati varieties in the group V gene pool, which is well separated from groups I and VI comprising of indica and japonica types, respectively³. Further evidence of high degree of divergence of Basmati from other indica varieties comes from high percentage of hybrid sterility⁴. The difficulties experienced in evolving ‘elite’ Basmati varieties combining all the desirable traits of TB and NB varieties have retained the preeminent status of TB varieties in the rice industry.

Consequently, TB varieties command considerable price advantage in the market over the EB varieties. The adulteration of TB grains with EB and NB grains is reported to be common and thus hampering the Basmati rice export market. Hence, identifying the genuine Basmati variety from the other Basmati-like non-Basmati varieties is considered to be important from the viewpoint of trade.

Traditionally employed morphological and chemical parameters have not been found to be discriminative enough warranting more precise techniques. Several molecular techniques are available for detecting genetic differences within and among cultivars⁵⁻⁸. Among these, Simple Sequence Repeat (SSR) markers are efficient and cost effective and detect a significantly higher degree of polymorphism in rice⁹⁻¹¹. They are ideal for genetic diversity studies and intensive genetic mapping¹²⁻¹⁴. An alternative method to SSRs, called Inter-SSR-PCR¹⁵ has also been used to fingerprint the rice varieties¹⁶.

The well-characterized Basmati rice specific molecular markers could serve as marker tags for Basmati varieties. If the markers are shown to be tightly linked to any of the distinct traits of Basmati they could be used in marker assisted selection (MAS) programs. Such markers could be further verified on the fully sequenced rice genome with regard to their location and linkage to the gene(s) of interest.

Recent progress in DNA marker technology, particularly PCR based markers, such as randomly amplified polymorphic DNA markers (RAPD),^{6, 17,18} amplified fragment length polymorphisms (AFLP),^{8,19} and microsatellite markers²⁰⁻²² have augmented the marker resources for genetic analyses of a wide variety of genomes. As PCR technology finds increased use in various genetic analyses, additional novel variations of this technique are emerging in order to augment the high-resolution genotyping and genetic mapping of various complex animal, plant and microbial genomes. The PCR analysis using anchored simple sequence repeat primers, referred to as ISSR-PCR or anchored SSR-PCR, has gained attention recently as an attractive means of characterizing complex genomes.^{15,16,17} The ISSR-PCR approach employs oligonucleotides based on simple sequence repeats (SSR) anchored either at the 5′ or 3′ end with two or four purine or pyrimidine residues, to initiate PCR amplification of genomic segments flanked by inversely oriented, closely spaced microsatellite repeats.¹⁵

The ISSR-PCR strategy is especially attractive because it avoids the need to carry out costly cloning and sequencing inherent in the original microsatellite-based approach. As a result, ISSR-PCR has been profitably used for genetic linkage analysis of various plant species²³⁻²⁷ and the silkworm, Bombyx mori.^{28,29}

The most commonly used approach for generating ISSR-PCR markers is either 5′-end labeling of ISSR primers with γ[³²P] ATP or one of the α[³²P] labeled dNTPs is added to the PCR reaction along with cold dNTPs in appropriate ratio, followed by resolution of PCR products on PAGE and autoradiographic detection of ISSR markers. Alternatively, some investigators have also resolved ISSR-PCR products on Nusieve agarose gels, of course with a marked reduction in number of markers compared to PAGE. While the former involves stringent standardization and extensive use of radioactive isotopes, the latter compromises with the number of markers generated per PCR reaction. Besides, both the methods require higher quantity (>10 ng) of template DNA per PCR reaction. These features prove to be disadvantageous in high resolution genetic mapping experiments where a large number of markers are analyzed using a single mapping population and genetic analysis of infectious organisms and parasites where DNA yield per sample may be too low for conventional PCR assays.

In the present study, the inventors have automated the ISSR-PCR marker assay to enhance genetic informativeness and used it along with rice SSRs to analyze the genetic relationships of traditional and evolved Basmati and non-Basmati varieties. Further, the inventors have designed and disclosed novel ISSR-PCR primers which have shown more resolving power than the known ISSR-PCR primers.

### OBJECTS OF THE PRESENT INVENTION

The main object of the present invention is to develop a set of inter-simple sequence repeats (ISSR)-PCR primers for genotyping eukaryotes.

Another main object of the present invention is to develop a method of genotyping diverse genomes of plant and animal systems using FISSR-PCR primers.

Yet another object of the present invention is to develop a FISSR method of distinguishing Basmati rice varieties from Non-Basmati (NB) rice varieties.

Still another object of the present invention is to develop a FISSR method of distinguishing Traditional Basmati (TB) rice varieties from Evolved Basmati (NB) rice varieties.

Still another object of the present invention is to develop a SSR method of distinguishing Basmati rice varieties from Non-Basmati (NB) rice varieties.

Still another object of the present invention is to develop a SSR method of distinguishing Traditional Basmati (TB) rice varieties from Evolved Basmati (NB) rice varieties.

Still another object of the present invention relates to develop a method of using the novel primers and markers in rice breeding to develop rice varieties of desired characteristics.

Still another main object of the present invention is to develop a method of genotyping animal systems including silkworm.

Still another object of the present invention is to develop a method of using the novel primers and markers to identify lineage of rice varieties.

Still another object of the present invention is to develop newer rice varieties of both Basmati and non-Basmati type.

Still another object of the present invention is to identify monomorphic, polymorphic, and diverse nature of the rice varieties using the said markers.

Still another object of the present invention is to develop a method of using simple sequence repeat (SSR) loci and corresponding 70 SSR alleles along with the said primers and markers to develop rice varieties of desired characteristics.

Another main object of the present invention is to determine adulteration of Basmati rice varieties with other kinds of rice varieties.

Yet another main object of the present invention is to develop a kit using FISSR-PCR primers to determine adulteration of Basmati rice varieties with other kinds of rice varieties.

Yet another main object of the present invention is to develop a kit using SSR markers to determine adulteration of Basmati rice varieties with other kinds of rice varieties.

### SUMMARY OF THE PRESENT INVENTION

The present invention relates to set of inter-simple sequence repeats (ISSR)-PCR primers of SEQ ID Nos. 1 to 37 for genotyping eukaryotes and a method of genotyping diverse genomes of plant and animal systems using FISSR-PCR primers and SSR markers; more particularly, a FISSR and SSR method of distinguishing Basmati rice varieties from Non-Basmati (NB) rice varieties, and Traditional Basmati (TB) rice varieties from Evolved Basmati (NB) rice varieties, and also, a method for determining adulteration of Basmati rice with other rice varieties and a kit thereof.

### DETAILED DESCRIPTION OF THE PRESENT INVENTION

Accordingly, the present invention relates to set of inter-simple sequence repeats (ISSR)-PCR primers of SEQ ID Nos. 1 to 37 for genotyping eukaryotes and a method of genotyping diverse genomes of plant and animal systems using FISSR-PCR primers; more particularly, a FISSR method of distinguishing Basmati rice varieties from Non-Basmati (NB) rice varieties, and Traditional Basmati (TB) rice varieties from Evolved Basmati (NB) rice varieties.

| | |
|---|---|
| GATGCTGATACACACACACACACA. | SEQ ID NO. 1 |
| GCACATGCAGTGTGTGTGTGTGTG | SEQ ID NO. 2 |
| CATGCACATTGTGTGTGTGTGTGT | SEQ ID NO. 3 |
| GCTAGTGCTCACACACACACACAC | SEQ ID NO. 4 |
| CGTATGTGTGTGTGTGTGTGTGT | SEQ ID NO. 5 |
| TGTAATGAGAGAGAGAGAGAGA | SEQ ID NO. 6 |
| GACGATACGAGAGAGAGAGAGA | SEQ ID NO. 7 |
| CCCGGGATTATTATTATT | SEQ ID NO. 8 |
| AAATACAGCAGCAGCAG | SEQ ID NO. 9 |
| GTGCTAATAATAATAAT | SEQ TD NO. 10 |
| AATTTATTATTATTATT | SEQ ID NO. 11 |
| GAGTCATTATTATTATT | SEQ ID NO. 12 |
| AGCGAATTATTATTATT | SEQ ID NO. 13 |
| TAAAAAATAATAATAAT | SEQ ID NO. 14 |
| ACAAAAATAATAATAAT | SEQ ID NO. 15 |
| AGTGAATTATTATTATT | SEQ ID NO. 16 |
| GTGATATTATTATTATT | SEQ ID NO. 17 |
| TGAGCGCCGCCGCCGCC | SEQ ID NO. 18 |
| TCGATACCACCACCACCACCACCACCA | SEQ ID NO. 19 |
| ATAGAGCTGCTGCTGCTGCTGCT | SEQ ID NO. 20 |
| AATCGAAAGAAGAAGAAG | SEQ ID NO. 21 |
| CATAATAAGAAGAAGAAG | SEQ ID NO. 22 |
| ATCGAATAATAATAATAATAAT | SEQ ID NO. 23 |
| GCATATGATGATGATG | SEQ ID NO. 24 |
| (A/T)T(G/C)GACAGACAGACAGACA | SEQ ID NO. 25 |
| GTGTGTGTGTGTGTGTATCC | SEQ ID NO. 26 |
| GAGAGAGAGAGAGAGACGG | SEQ ID NO. 27 |
| AAGAAGAAGAAGAACTA | SEQ ID NO. 28 |
| AAGAAGAAGAAGTACGA | SEQ ID NO. 29 |
| CTTCTTCTTCTTATGCT | SEQ ID NO. 30 |
| GGCGGCGGCGGCGCTAA | SEQ ID NO. 31 |
| ATGATGATGATGGACT | SEQ ID NO. 32 |
| AAACAAACAAACATC | SEQ ID NO. 33 |
| CACACACACACACAATGCACAGC | SEQ ID NO. 34 |
| GAGAGAGAGAGAGAACTAT | SEQ ID NO. 35 |
| GCCGCCGCCGCCGCACTC | SEQ ID NO. 36 |
| AACAACAACAACGT | SEQ ID NO. 37 |

In an embodiment of the present invention, wherein a set of inter-simple sequence repeats (ISSR)-PCR primers of SEQ ID Nos. 1 to 37 for genotyping eukaryotes. In another embodiment of the present invention, wherein primers of SEQ ID No. 1 to 25 are 5′ anchored primers.

In yet another embodiment of the present invention, wherein primers of SEQ ID Nos. 26 to and 37 are 3′ anchored primers.

In another embodiment of the present invention, wherein a method of genotyping diverse genomes of plant and animal systems using the said FISSR-PCR primers, said method comprising steps of:

(a). extracting DNA from said systems,

(b). conducting a polymerase chain reaction (PCR) using extracted DNA, the said primers, and a flourescent label,

(c). obtaining a plurality of flourescent amplified products,

(d). separating the amplified products to produce fingerprint pattern using conventional techniques,

(e). identifying Monomorphism (M), and Polymorphism (P) amplified products, and

(f). genotyping the genomes of the said systems based on the polymorphic amplified fragments.

In an embodiment of the present invention, wherein the flourescent label can be selected from a group comprising Tamara dye, R6G, and R110.

In yet another embodiment of the present invention, wherein a FISSR method of distinguishing Basmati rice varieties from Non-Basmati (NB) rice varieties, using primers of SEQ ID Nos. 1-5, 7, 11, 19, 20, 25, 26, and 27, said method comprising steps of:

(a). extracting DNA from said rice varieties,

(b). conducting a polymerase chain reaction (PCR) using extracted DNA, the said primers, and a flourescent label,

(c). obtaining a plurality of flourescent amplified products,

(d). separating the amplified products to produce fingerprint pattern using conventional techniques,

(e). identifying Monomorphism (M), and Polymorphism (P) amplified products, and

(f). distinguishing Basmati rice varieties from Non-Basmati (NB) rice varieties based on the polymorphic amplified fragments

In still another embodiment of the present invention, wherein the flourescent label can be selected from a group comprising Tamara dye, R6G, and R110.

In another embodiment of the present invention, wherein a FISSR method of distinguishing Traditional Basmati (TB) rice varieties from Evolved Basmati (NB) rice varieties, using primers of SEQ ID Nos. 1-5, 7, 11, 19, 20, 25, 26, and 27, said method comprising steps of:

(a). extracting DNA from said rice varieties,

(b). conducting a polymerase chain reaction (PCR) using extracted DNA, the said primers, and a flourescent label,

(c). obtaining a plurality of flourescent amplified products,

(d). separating the amplified products to produce fingerprint pattern using conventional techniques,

(e). identifying Monomorphism (M), and Polymorphism (P) amplified products, and

(f). distinguishing Traditional Basmati (TB) rice varieties from Evolved Basmati (EB) rice varieties based on the polymorphic amplified fragments

In yet another embodiment of the present invention, wherein the flourescent label can be selected from a group comprising Tamara dye, R6G, and R110.

In still an embodiment of the present invention, wherein the average number of bands produced by the primers with different repeat motifs negatively correlated with the number of nucleotides in the repeat unit of the motif.

In still another embodiment of the present invention, wherein the number of products amplified in different repeat length classes reflect the frequency of different repeat motifs distributed in the rice genome.

In another main embodiment of the present invention, wherein a method of genotyping diverse genomes of plant and animal systems using SSR-PCR markers of table 3, said method comprising steps of:

(a). extracting DNA from said systems,

(b). conducting a polymerase chain reaction (PCR) using extracted DNA, the said primers, and a flourescent label,

(c). obtaining a plurality of flourescent amplified products,

(d). separating the amplified products to produce fingerprint pattern using conventional techniques,

(e). identifying Monomorphism (M), and Polymorphism (P) amplified products, and

(f). genotyping the genomes of the said systems based on the polymorphic amplified fragments.

In yet another embodiment of the present invention, wherein the flourescent label can be selected from a group comprising Tamara dye, R6G, and R110.

In still another embodiment of the present invention, wherein a SSR method of distinguishing Basmati rice varieties from Non-Basmati (NB) rice varieties, using markers of table 4, said method comprising steps of:

(a). extracting DNA from said rice varieties,

(b). conducting a polymerase chain reaction (PCR) using extracted DNA, the said primers, and a flourescent label,

(c). obtaining a plurality of amplified products,

(d). separating the amplified products to produce fingerprint pattern using conventional techniques,

(e). identifying Monomorphism (M), and Polymorphism (P) amplified products, and

(f). distinguishing Basmati rice varieties from Non-Basmati (NB) rice varieties based on the polymorphic amplified fragments

In still another embodiment of the present invention, wherein the flourescent label can be selected from a group comprising Tamara dye, R6G, and R110.

In still another embodiment of the present invention, wherein a SSR method of distinguishing Traditional Basmati (TB) rice varieties from Evolved Basmati (NB) rice varieties, using markers of Table 5, said method comprising steps of:

(a). extracting DNA from said rice varieties,

(b). conducting a polymerase chain reaction (PCR) using extracted DNA, the said primers, and a flourescent label,

(c). obtaining a plurality of flourescent amplified products,

(d). separating the amplified products to produce fingerprint pattern using conventional techniques,

(e). identifying Monomorphism (M), and Polymorphism (P) amplified products, and

(f). distinguishing Traditional Basmati (TB) rice varieties from Evolved Basmati (EB) rice varieties based on the polymorphic amplified fragments

In still another embodiment of the present invention, wherein the flourescent label can be selected from a group comprising Tamara dye, R6G, and R110.

In another main embodiment of the present invention, wherein a kit for determining adulteration of Basmati rice with other rice varieties, said kit comprising

(a). at least one ISSR-PCR primers from a set of primers of SEQ ID Nos. 1 to 37, and/or

(b). at least one SSR markers from a set of markers of Table-4.

In yet another main embodiment of the present invention, wherein a method for determining adulteration of Basmati rice with other rice varieties (FIG. 4) using at least one ISSR-PCR primers from a set of primers of SEQ ID Nos. 1 to 37, and/or at least one SSR markers from a set of markers of Table-4, said method comprising steps of:

(a). extracting DNA from various rice varieties,

(b). conducting a polymerase chain reaction (PCR) using extracted DNA, the said primer(s) or marker(s), and a flourescent label,

(c). obtaining a plurality of flourescent amplified products,

(d). separating the amplified products to produce fingerprint pattern using conventional techniques,

(e). identifying Monomorphism (M), and Polymorphism (P) amplified products, and

(f). determining adulteration in Basmati rice varieties with other rice varieties based on the polymorphic amplified fragments.

In still another embodiment of the present invention, wherein accordingly, the present invention relates to 37 inter-SSR-PCR primers and corresponding 481 inter-SSR-PCR markers useful in revealing genetic relationship in Basmati and non-Basmati rice varieties and a method of using the said primers, markers, and selected simple sequence repeat (SSR) loci and corresponding SSR alleles in rice breeding, for developing rice varieties of desired characteristics and also, a method of using the said primers, markers, SSR loci and SSR alleles in identifying genetic lineage of rice varieties.

In still another embodiment of the present invention, wherein the recently developed Inter-Simple Sequence Repeat PCR (ISSR-PCR) or microsatellite primed PCR or Simple Sequence Repeat (SSR)-Anchored PCR technique detects polymorphic markers in a wide variety of genomes. Usually the ISSR primers are either 5′ end-labeled with γ[³²P]ATP or one of the α[³²P] labeled dNTPs is added to the PCR reaction and the PCR products are resolved on PAGE and autoradiographed. Alternatively, cold PCR products are resolved on agarose gel electrophoresis. In the present study, we show that informativity, sensitivity and speed of the ISSR-PCR can be substantially enhanced by adding fluorescent nucleotide in the PCR reaction followed by resolution of PCR products on an ABI 377 automated sequencer. The informativeness, measured as a number of detectable amplified fragments, was two-fold higher and the quantity of required template DNA is two-fold lower than the regular ISSR-PCR. We have termed this method as FISSR-PCR and show its usefulness in generating large number of species and varietal specific markers in plants, insects, parasites of insects and human and various infectious organisms. Further, we show that the FISSR markers are inherited and segregated in Mendelian fashion as demonstrated on a panel of 99 F₂ offspring derived from a cross of two divergent silkworm strains. The FISSR-PCR marker assay could be a method of choice for large scale screening of varieties/cultivers and highthroughput genotyping in mapping of genomes where microsatellite information is scanty or absent.

In still another embodiment of the present invention, wherein the objective of the present study was to make use of the efficient molecular marker systems to reveal genetic relationships in traditional Basmati (TB) and evolved Basmati (EB) and semi-dwarf non-Basmati (NB) rice varieties. A subset of three rice groups was analyzed using 19 SSR loci and 37 (Inter simple Sequence Repeat) ISSR-PCR primers. A total of 70 SSR (Simple Sequence Repeat) alleles and 481 ISSR-PCR markers were revealed in 24 varieties from the three groups. The lowest genetic diversity was observed among the traditional Basmati varieties whereas the evolved Basmati varieties showed the highest genetic diversity by both the marker assays. The results indicated that the subset of aromatic rice varieties analyzed in the present study is probably derived from a single land race. The traditional Basmati and semi-dwarf non-Basmati rice varieties used in the present study were clearly delineated by both the marker assays. A number of markers, which could unambiguously distinguish the traditional Basmati varieties used in the present study from the evolved ones and non-Basmati rice varieties, were identified. The potential use of these markers in Basmati rice breeding program and authentication of traditional Basmati varieties used in the present study is envisaged.

In still another embodiment of the present invention, wherein the certified Basmati rice materials used in this study were provided by the Ministry of Commerce, Govt. of India and non-Basmati varieties by the Directorate of Rice Research, Hyderabad, India. The details of the rice varieties are given in Table 1 as shown here below.

TABLE 1
List of rice varieties (Oryza sativa) included in the study

| Name | Origin |
|---|---|
| Traditional Basmati (TB) | |
| Basmati 217 | Punjab (Indian sub-continent) |
| Basmati 370 | Punjab (Indian sub-continent) |
| Dehraduni (Type-3) | Uttar Pradesh |
| Ranbir Basmati | Jammu & Kashmir |
| Tarori (HBC-19) | Haryana |
| Basmati 386 | India |
| Evolved Basmati (ER) | |
| Basmati 385 (385) | TN-1 × Basmati 370 |
| Super Basmati (SB) | Basmati 320 × IR 661 |
| Pusa Basmati (PB) | Pusa 150 × Karnal local |
| Kasturi (Kas) | CK 88-17-1-5 × Basmati 370 |
| Haryana Basmati (HB) | Sona × Basmati 370 |
| Mahi Sugantha (MS) | BK 79 × Basmati 370 |
| Haryana Gaurav (HG) | Mutant of Basmati 370 |
| Super (SU) | Equivalent to SB |
| Terricot (TER) | NA |
| Sharbati (SHA) | NA |
| CSR 30B (CSR) | Buraratha 4-10 × Pak Basmati |
| | ? |
| Semi-dwarf non Basmati | |
| (NB) | |
| IR 8 | Pesa × Dee-geoe- |
| Jaya | woo-gen |
| Taichung (N) 1 (TC) | TN 1 × T 141 |
| IR 22 | NA |
| IR 20 | IR 8 × Tadukan |
| IR BB5 | IR 262 × TKM 6 |
| PR 106 (PR) | IR 24 × DZ 192 |
| | IR 8 × Peta 5 × Bella patna |

Varietal abbreviations are indicated in the parentheses

In still another embodiment of the present invention, wherein the DNA was extracted from 5 g of grains from each of the varieties using Phytopure plant DNA extraction kit (Pharmacia Amersham Biotech).

In still another embodiment of the present invention, wherein DNA markers and laboratory assay. Two classes of markers were employed in the present study: fluorescence based ISSR-PCR and SSRs.

ISSR-PCR. The ISSR-PCR method (15) was modified with a view to enhance the speed and sensitivity of detection of markers. We designed and synthesized 12 5′ and 3′ anchored primers (Please refer Table 2 here below).

TABLE 2
List of ISSR primers, marker information and diversity in three rice groups

| | | | Total No. | No. of markers | | | | | | | | |
|---|---|---|---|---|---|---|---|---|---|---|---|---|
| | | Mol. Wt | of | TB | | | EB | | | NB | | |
| Primers | Sequences | Range (bp) | markers | M | P | D | M | P | D | M | P | D |

5′ anchored

| | | | | | | | | | | | | |
|---|---|---|---|---|---|---|---|---|---|---|---|---|
| C (GA)₇ | SEQ ID NO.7 | 150-1200 | 50 | 31 | 11 | 0.18 | 10 | 40 | 0.56 | 15 | 22 | 0.43 |
| R (CA)₇ | SEQ ID NO.1 | 150-1500 | 45 | 20 | 9 | 0.27 | 14 | 27 | 0.51 | 24 | 18 | 0.23 |
| T (CA)₇ | SEQ ID NO.4 | 180-1200 | 38 | 17 | 6 | 0.16 | 6 | 30 | 0.68 | 16 | 9 | 0.21 |
| T (GT)₉ | SEQ ID NO.5 | 200-1500 | 61 | 29 | 11 | 0.18 | 6 | 53 | 0.67 | 12 | 21 | 0.42 |
| R (TG)₇ | SEQ ID NO.2 | 160-1400 | 42 | 23 | 6 | 0.15 | 14 | 27 | 0.49 | 14 | 16 | 0.29 |
| Y (TG)₇ | SEQ ID NO.3 | 200-1050 | 33 | 14 | 5 | 0.18 | 7 | 26 | 0.63 | 11 | 13 | 0.34 |
| T3(ATT)₄ | SEQ ID NO.11 | 250-1500 | 50 | 3 | 32 | 0.60 | 2 | 47 | 0.72 | 2 | 22 | 0.65 |
| RA(GCT)₆ | SEQ ID NO.20 | 180-760 | 30 | 20 | 4 | 0.07 | 11 | 18 | 0.37 | 13 | 7 | 0.24 |
| Y (ACC)₇ | SEQ ID NO.19 | 200-960 | 33 | 19 | 6 | 0.17 | 9 | 22 | 0.51 | 13 | 7 | 0.19 |
| (GACA)₄ | SEQ ID NO.25 | 315-1400 | 18 | 7 | 7 | 0.30 | 4 | 14 | 0.59 | 5 | 10 | 0.60 |
| 3′ anchored | | | | | | | | | | | | |
| (GA)₈C | SEQ ID NO.27 | 150-1150 | 49 | 28 | 10 | 0.17 | 18 | 30 | 0.45 | 20 | 13 | 0.23 |
| (GT)₈R | SEQ ID NO.26 | 140-960 | 32 | 14 | 8 | 0.27 | 4 | 26 | 0.63 | 10 | 8 | 0.28 |
| Total | | | 481 | | | 0.23 ± | | | 0.57 ± | | | 0.3 ± |
| | | | | | | 0.13 | | | 0.10 | | | 0.15 |

M: Monomorphic;
P: Polymorphic;
D: Diversity

In still another embodiment of the present invention, wherein amplification was performed in 10 mM Tris-HCl, pH 8.3 (50 mM KCl; 1.5 mM MgCl₂; 0.01% gelatin; 0.01% Triton X-100), 1 mM dNTPs, 0.2 μM Fluorescent dUTP (TAMARA, Perkin Elmer), 0.3 unit of AmpliTaq Gold (Perkin Elmer), 4 μM primer with 5 ng of genomic DNA per 5 μl reaction. The thermal cycling conditions were as follows: initial denaturation of 10 min at 94° C., 35 cycles of 30 s at 94° C., 30 s at 50° C. and 1 min at 72° C., final extension of 10 min at 72° C. The PCR was performed on a Perkin Elmer thermal cycler (9600). One μl of PCR product was mixed with 1.5 μl of 6× loading buffer (1:4 mixture of loading buffer and formamide, Sigma Chemicals Co.) and 0.4 μl of GENESCAN-1000 ROX labelled molecular weight standard (red fluorescence) was included in the loading samples. The samples were denatured at 92° C. for 1 min prior to loading onto an ABI 377 automated sequencer (Applied Biosystems) and electrophoresed on 5% polyacrylamide gel (Long ranger, FMC) under denaturing conditions containing 7 M urea, in 1×TBE buffer (90 mM Tris borate, pH 8.3 and 2 mM EDTA). Three replicate experiments were carried out to verify the reproducibility of markers.

In still another embodiment of the present invention, wherein Selection of primers and SSR survey. We selected 19 primer pairs (Please refer Table 3 here below) from the list of 351 rice microsatellite loci displayed on the Cornell university RiceGenes website (http://ars-genome.cornell.edu/rice/) for analysis.

TABLE 3
Microsatellite marker information, allele distribution and diversity in three rice groups

| | | Total | | No. of alleles | | | | | | | | |
|---|---|---|---|---|---|---|---|---|---|---|---|---|
| | | No. of | Allele Size | TB | | | EB | | | NB | | |
| Locus | Repeat motif | Alleles | range (bp) | M | P | D | M | P | D | M | P | D |
| RM 224 | (GA)₁₃ | 4 | 130-155 | 1 | 1 | 0.49 | 0 | 3 | 0.74 | 0 | 4 | 0.91 |
| RM 16 | (GA)₁₅ | 3 | 165-225 | 1 | 0 | 0.00 | 0 | 2 | 0.95 | 0 | 2 | 0.83 |
| RM 13 | (GA)₁₆ | 6 | 135-158 | 0 | 2 | 0.62 | 0 | 4 | 0.91 | 0 | 4 | 0.86 |
| RM 252 | (GA)₁₉ | 8 | 195-255 | 0 | 4 | 0.86 | 0 | 4 | 0.92 | 0 | 3 | 0.82 |
| RM 235 | (GA)₂₄ | 3 | 102-138 | 0 | 2 | 0.62 | 0 | 2 | 0.74 | 0 | 3 | 0.82 |
| RM 234 | (GA)₂₅ | 2 | 148-150 | 1 | 0 | 0.00 | 0 | 2 | 0.75 | 1 | 0 | 0.00 |
| RM 223 | (GA)₂₅ | 4 | 170-180 | 1 | 0 | 0.00 | 0 | 4 | 0.88 | 1 | 0 | 0.00 |
| RM 1 | (GA)₂₆ | 3 | 85-110 | 1 | 0 | 0.00 | 0 | 3 | 0.86 | 0 | 2 | 0.38 |
| RM 310 | (GT)₁₉ | 5 | 85-110 | 0 | 2 | 0.27 | 0 | 4 | 0.92 | 0 | 3 | 0.88 |
| RM 302 | (GT)₃₀(AT)₈ | 4 | 136-205 | 1 | 0 | 0.00 | 0 | 4 | 0.93 | 0 | 2 | 0.62 |
| RM 160 | (GAA)₂₃ | 2 | 100-105 | 1 | 0 | 0.00 | 0 | 2 | 0.71 | 1 | 0 | 0.00 |
| RM 330 | (CAT)₅ | 5 | 160-225 | 3 | 0 | 0.00 | 1 | 3 | 0.42 | 1 | 3 | 0.26 |
| RM 72 | (TAT)₅C(ATT)₁₅ | 3 | 150-175 | 0 | 2 | 0.62 | 0 | 3 | 0.89 | 0 | 2 | 0.82 |
| RM 102 | (GGC)₇(CG)₆ | 3 | 435-445 | 2 | 0 | 0.00 | 0 | 3 | 0.58 | 1 | 2 | 0.42 |
| RM 171 | (GATG)₅ | 3 | 325-346 | 0 | 2 | 0.49 | 0 | 2 | 0.77 | 0 | 2 | 0.55 |
| RM 163 | (GGAGA)₄ | 4 | 130-175 | 1 | 0 | 0.00 | 0 | 3 | 0.86 | 0 | 4 | 0.89 |
| | (GA)₁₁C(GA)₂₀ | | | | | | | | | | | |
| RM 161 | (AG)₂₀ | 3 | 163-180 | 1 | 1 | 0.49 | 0 | 2 | 0.74 | 1 | 0 | 0.00 |
| RM 136 | (AGG)₇ | 3 | 100-124 | 1 | 0 | 0.00 | 0 | 2 | 0.71 | 1 | 1 | 0.49 |
| RM 238 | NA | 2 | 130-150 | 1 | 0 | 0.00 | 0 | 2 | 0.85 | 1 | 0 | 0.00 |
| Total | | 70 | | 16 | 16 | 0.23 ± | 1 | 54 | 0.79 ± | 8 | 37 | 0.50 ± |
| No. of | | | | | | 0.30 | | | 0.13 | | | 0.36 |
| alleles | | | | | | | | | | | | |

M: Monomorphic;
P: Polymorphic;
D: Diversity

In still another embodiment of the present invention, wherein the SSR primers are obtained from the databank. The details of the Accession numbers are as given below.

1. RM 171—D84275

2. RM 161—D41873

3. RM 72—AF344086

4. RM 13—AF344014

5. RM 238A—AF 344058

6. RM 16—AF 344016

7. RM 330—AF 344154

8. RM 302—AF 344127

9. RM 224—AF 344045

10. RM 252—AF 344072

11. RM 234—AF 344054

12. RM 223—AF 344044

13. RM 102—D 17586

14. RM 136—RM 136

In still another embodiment of the present invention, wherein the primers for the selected loci were synthesized by Research Genetics (Huntsville, Ala). Wherever possible at least two loci with non-overlapping alleles were multiplexed in the PCR reaction to increase the efficiency of geno typing.

In still another embodiment of the present invention, wherein PCR amplification was performed in a 5 μl volume containing 10 mM Tris-HCl (pH 8.3), 50 mM KCl, 1.5 mM MgCl₂, 0.25 unit of AmpliTaq Gold (Perkin Elmer), 50 μM of dNTPs, 0.2 μM fluorescent dUTP (TAMARA or R110 or R6G, Perkin Elmer), 0.35 μM of each primer with 5 ng of genomic DNA on a Themal Cycler PTC 100 (MJ Research Inc.). The basic PCR program used to amplify the SSR DNA was as described in the RiceGenes website. The sample preparation, loading and electrophoretic conditions were as described under ISSR-PCR. When the allelic polymorphisms of any of the microsatellite loci revealed >20 bp difference between the varieties, such loci were resolved on 3.5% MetaPhor agarose gels (FMC).

In still another embodiment of the present invention, wherein evaluation of polymorphisms and data analysis. Polymorphic products from the SSR and ISSR analyses were scored qualitatively for presence (+) or absence (−). The proportion of bands that were shared between any of the two varieties screened averaged over loci (SSRs) and primers (ISSR-PCR) were used as the measure of similarity. The genetic diversity⁵ was calculated as follows:

$${PIC}_{i} = 1 - {\sum }_{j = 1}^{n} {P}_{ij}^{2}$$

where Pij is the frequency of the ^{jth} allele for marker i and the summation extends over n alleles. The calculation was based on the number of alleles/locus in SSR, and number of bands/primer in case of ISSR.

In still another embodiment of the present invention, wherein Cluster analysis was based on distance matrices using the unweighted pair group method analysis (UPGMA) program in WINBOOT software³¹. The relationships between varieties were represented graphically in the form of dendrograms.

In still another embodiment of the present invention, wherein it has been shown that by using DNA markers identified in the present study, the detection of adulteration of hybrid Basmati with Pure Basmati could be detected upto the level of 1% adulterant in the mixture (FIG. 4).

### BRIEF DESCRIPTION OF THE ACCOMPANYING DRAWINGS

FIG. 1 shows Fluorescence based ISSR profiles of TB, EB and NB rice varieties for three representative primers: a) SEQ ID NO. 5; b) SEQ ID NO. 3; c) SEQ ID NO. 26. Arrows and arrowheads indicate the markers, which differentiate Basmati and non-Basmati rice varieties respectively.

FIG. 2 shows Fluorescence based SSR polymorphisms detected by 8 representative SSR loci in TB, EB and NB rice varieties. Arrows and arrowheads indicate the markers, which differentiate Basmati and non-Basmati rice varieties respectively. The asterisk indicates the duplication of locus 330 only in NB and some of the EB varieties. The first 7 loci are GENESCAN images and the last locus (RM 234) is 3.5% Metaphor agarose run ethidium bromide stained image.

FIG. 3 shows Dendrogram derived from a UPGMA cluster analysis using Nei and Li Coefficients based on (a) ISSR markers; (b) SSR markers. Numbers on the nodes indicate the number of times a particular branch was recorded per 100 bootstrap replications following 1000 replications.

FIG. 4 shows the method for determining adulteration of Basmati rice with other rice varieties.

FIGS. 5(a-c). Comparison of regular ISSR-PCR and FISSR-PCR assays using the primer 5′ CRT RT(GT)₉ 3′ (i.e. SEQ. ID. NO.5) in eight crop species on three electrophoretic systems showing reduced sensitivity on Nusieve agarose (a); intermediate on polyacrylamide sequencing gel with radiolabeling (b), and high sensitivity in FISSR-PCR products resolved on an ABI 377 sequencer (c).

FIG. 6 shows The Mendelian segregation of FISSR markers in silkworm. The FISSR markers were generated using a primer 5′ RAY RAT RC(GA)₇ 3′ on two parental strains, P⁵⁰ and C₁₀₈, and their F₁ and F₂ offspring. The arrows and arrowheads indicate markers specific to P⁵⁰ and C₁₀₈, respectively.

FIG. 7 shows that the ISSR-PCR markers can be used to identify the varieties. Here, Lane 1 represents an elite breed of tomato. Lanes 2 and 3 represents tomato breeds sold in the name of the elite breed profiled in Lane 1.

FIG. 8 shows FISSR-PCR profiles of different clones of Casuarina. Here, A stands for Allocasurina.

FIG. 9 shows the DNA profiles of various micro-organisms using FISSR primer.

FIG. 10 shows the DNA profiles of different species of silkmoths using FISSR primer

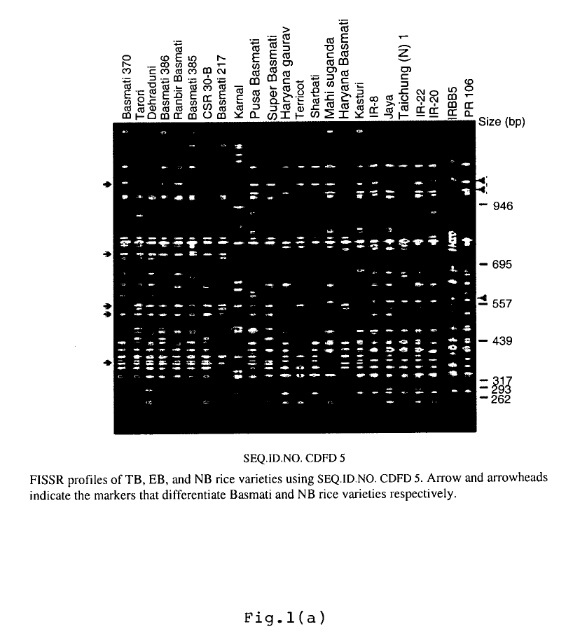

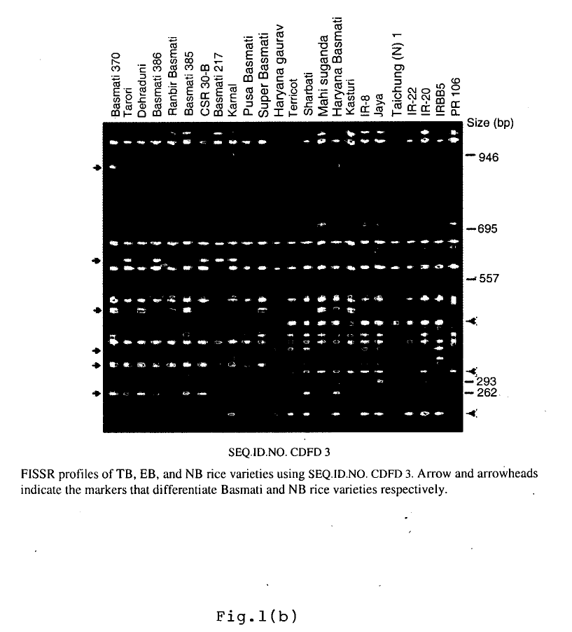

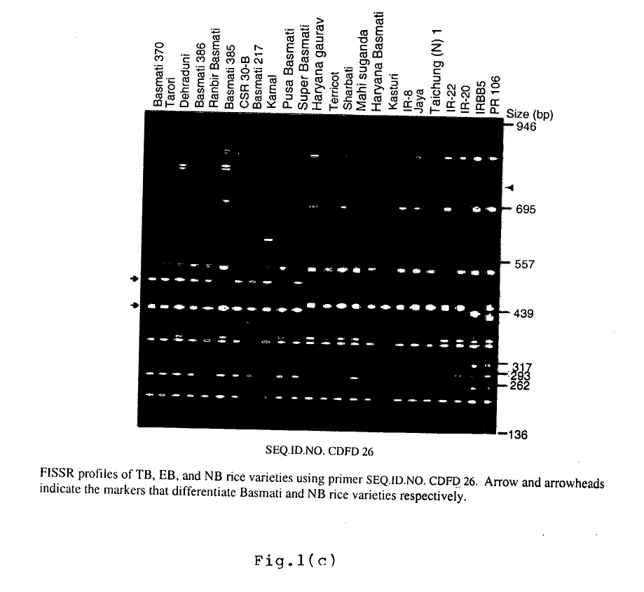

TABLE 4
A sample of FISSR markers that distinguish
Basmati and Non-Basmati rice varieties
(see FIG. 1)

| | | | Non |
|---|---|---|---|
| Primers | Size(bp) | Basmati | Basmati |
| CGTAT(GT)₉ | 377 | + | − |
| | 550 | + | − |
| | 558 | + | − |
| | 571 | − | + |
| | 743 | + | − |
| CATGCACAT(TG)₇T | 208 | − | + |
| | 262 | + | − |
| | 326 | − | + |
| | 345 | + | − |
| | 378 | + | − |
| | 448 | − | + |
| | 480 | + | − |
| | 611 | + | − |
| | 908 | + | − |
| (GT)₈ATCC | 436 | + | − |
| | 497 | + | − |
| | 790 | − | + |

TABLE 5
A sample of FISSR markers that distinguish
traditional and evolved Basmati rice varieties

| | | Pure | |
|---|---|---|---|
| Primers | Size(bp) | Basmati | Evolved Varieties |
| CGTAT(GT)₉ | 1200 | + | −(except CSR, SU) |
| | 730 | + | −(except SU) |
| | 750 | + | −(except SU, SB) |
| | 550 | + | −(except CSR, SU, PB, 385) |
| CATGCACAT(TG)₇T | 908 | + | −(except CSR, SU, PB, 385) |
| | 378 | + | −(except CSR, SB) |
| | 326 | + | −(except CSR, SU, SB, PB, |
| | | | 385) |
| | 262 | + | −(except CSR, SB, SH, HB) |
| | 208 | − | +(Except CSR, SU, 385, |
| | | | MS, KAS) |
| (GT)₈ATCC | 436 | + | −(except CSR, SU, SB, |
| | | | PB, 385) |
| | 443 | − | +(except CSR, SU, SB, PB, |
| | | | 385) |
| | 497 | + | −(except CSR, SU, SB, 385) |
| | 790 | − | +(except CSR, SU, SB, 385) |
| | 830 | + | −(except CSR, SU, SB, PB, |
| | | | 385) |

In still another embodiment of the present invention, wherein evaluation of ISSR-PCR markers. The fluorescence based ISSR-PCR markers could be clearly resolved on an ABI automated sequencing gels. All the 12 anchored SSR motifs employed in the present study produced varying number of DNA fragments in different size range (Table 2, FIG. 1). The average number of bands produced by ISSR primers with different repeat motifs was negatively correlated with the number of nucleotides in the repeat unit of the motif. For example, among the 5′ anchored primers, the dinucleotide-based primers produced more number of bands (43.8±9.82) than tri- (37.6±7.89) and tetranucleotide (32) primers. Although we have not used similar number of primers in each repeat class of primers for such a comparison, the number of products amplified in different repeat length class reflected the frequency of different repeat motifs distributed in the rice genome. The 12 primers produced a total of 481 PCR products, of which 389 (80.9%) were polymorphic in all the 24 rice varieties. There appeared to be no correlation between the number of bands amplified and the degree of polymorphisms. For example, the primers R(TG)₇ and Y(TG)₇ generated 42 and 33 bands respectively, out of which 73.8% and 78.8% were polymorphic, on the other hand 98% of the 50 bands amplified by T3(ATT)₄ primer were polymorphic. Among the 12 primers, two 5′ anchored, T3(ATT)₄ and T(GT)₉ and one 3′ anchored primer, (GT)₈R generated more than 90% scorable polymorphisms.

In still another embodiment of the present invention, wherein since our efforts were directed towards comparative genetic analysis of TB, EB and NB rice varieties, we evaluated the polymorphisms in these three groups. The degree of polymorphisms differed substantially among the three groups. The TB varieties showed very low level of polymorphism as compared to EB and NB varieties. Out of 340 bands scored in the TB varieties, only 115 (33.8%) were polymorphic (Table 2). On the other hand, 360 (77.4%) out of 465 bands were polymorphic in the EB and 166 (51.7%) out of 321 bands were polymorphic in NB varieties. The primer T3(ATT)₄ was found to produce the most polymorphic bands in all the three rice groups: TB (91.4%), EB (92%) and NB (91.66) whereas RA(GCT)₆ generated the least degree of polymorphisms (Table 2).

In still another embodiment of the present invention, wherein the genetic diversity was calculated for each of the ISSR primers in all the three rice groups (Table 2). The difference in diversity values was as striking as the degree of polymorphism. The two primers, T3(ATT)₄ and RA(GCT)₆ which recorded the highest and the lowest polymorphisms respectively, in the three groups also recorded the highest and the lowest diversity values. The 12 primers had an average diversity value of 0.23±0.132 with a range of 0.07 to 0.6 for TB. The diversity values ranged from 0.37 to 0.72 averaging 0.57±0.10 for the EB, and 0.19 to 0.65 with a mean of 0.34±0.15 for the NB varieties. The difference was significant (p<0.001, between TB and EB; p<0.10 between TB and NB groups). Out of the 12 ISSR-PCR primers, 5 revealed 12 PCR products, a combination of which could distinguish Basmati from NB varieties (data not shown). On the other hand 21 specific PCR products generated by 9 ISSR-PCR primers could distinguish EB from NB varieties (FIGS. 1a to 1c). By making use of 2 to 3 informative primers the TB from the EB and NB varieties could be unambiguously distinguished. For example, by using two primers SEQ ID NO. 5, and SEQ ID NO. 26 (FIGS. 1a-and 1c) one can make a decision whether a given variety is a TB or an EB rice variety.

In still another embodiment of the present invention, wherein evaluation of SSR polymorphisms. We also used 19 microsatellite loci for the genetic analysis of the three rice groups. Table 3 summarizes the total number of alleles detected and their size range across 24 rice varieties for each of the 19 microsatellite loci used. The number of alleles ranged from 2 to 8 with an average of 3.8 alleles. Only RM 252 detected a maximum of 8 alleles. As could be seen from Table 3 there appears to be no correlation between the number of alleles detected and the number of SSR repeats in the SSR loci. For example, the microsatellite loci containing the (GA) repeat motifs varying from (GA)₁₅ to (GA)₂₅ did not show any correlation with the number of alleles they revealed. The allele number varied between the three rice groups (Table 3). More number of alleles were resolved in the EB (56) and NB (42) as compared to TB (28) varieties. Out of 19 loci, only 8 revealed polymorphisms in the TB varieties whereas 14 were polymorphic in the NB varieties and all the loci were polymorphic in the EB varieties (Table 3). The diversity values also varied from one locus to another and between the three rice groups (Table 3). An average diversity of 0.23±0.3 was observed for the TB group whereas EB and NB rice groups recorded much higher diversity values of 0.79±0.13 and 0.50±0.36, respectively. However, the extent of variation in average diversity is not as large as the difference in the number of alleles per locus and it does not appear to correlate with the number of alleles. Across the three rice groups, the highest diversity of 0.87 was observed for RM 252 with 8 alleles whereas RM 72 and RM 16 with 3 alleles each displayed diversity of 0.70 and 0.76, respectively. The difference in diversity between the 3 groups was statistically significant (p<0.001, between TB and EB; p<0.05, between TB and NB groups).

In still another embodiment of the present invention, wherein we scored the SSR alleles which showed preponderance in Basmati varieties. Out of 70 alleles, 9 were found only in TB and in some of the EB varieties. These alleles were absent in all the 7 NB varieties analyzed in the present study (Please refer Table 6 here below)

TABLE 6
SSR loci that distinguish Basmati and semi dwarf rice varieties

| | | | Allele | | |
|---|---|---|---|---|---|
| | | | Size | Basmati | |
| LOCUS | Forward primer | Reverse primer | (bp) | varieties | Semi dwarf varieties |
| RM 163 | ATCCATGTGCGCCTTTATGAGGA | CGCTACCTCCTTCACTTACTAGT | 162 | − | +(only PR, TC(N)) |
| RM 171 | AACGCGAGGACACGTACTTAC | ACGAGATACGTACGCCTTTG | 438 | + | − |
| RM 161 | TGCAGATGAGAAGCGGCGCCTC | TGTGTCATCAGACGGCGCTCCG | 180 | + | − |
| RM 72 | CCGGCGATAAAACAATGAG | GCATCGGTCCTAACTAAGGG | 175 | + | − |
| RM 1 | GCGAAAACACAATGCAAAAA | GCGTTGGTTGGACCTGAC | 85 | − | |
| RM 13 | TCCAACATGGCAAGAGAGAG | GGTGGCATTCGATTCCAG | 158 | − | +(only PR) |
| RM 238 A | GATGGAAAGCACGTGCACTA | ACAGGCAATCCGTAGACTCG | 445 | + | − |
| RM 16 | CGCTAGGGCAGCATCTAAAA | AACACAGCAGGTACGCGC | 225 | − | +(only JA) |
| ″ | ″ | ″ | 165 | + | − |
| RM 330 | CAATGAAGTGGATCTCGGAG | CATCAATCAGCGAAGGTCC | 220 | + | − |
| ″ | ″ | ″ | 160 | − | +(except IR8, PR) |
| RM 302 | TCATGTCATCTACCATCACAC | ATGGAGAAGATGGAATACTTGC | 140 | + | − |
| RM 224 | ATCGATCGATCTTCACGAGG | TGCTATAAAAGGCATTCGGG | 148 | − | (only PR) |
| RM 252 | TTCGCTGACGTGATAGGTTG | ATGACTTGATCCCGAGAACG | 255 | − | (only IR20) |
| ″ | ″ | ″ | 228 | − | +(only TC(N)) - |
| RM 234 | ACAGTATCCAAGGCCCTGG | CACGTGAGACAAAGACGGAG | 148 | + | − |
| RM 223 | GAGTGAGCTTGGGCTGAAAC | GAAGGCAAGTCTTGGCACTG | 200 | + | − |

In still another embodiment of the present invention, wherein further analysis of seven more NB varieties confirmed that these 9 alleles are confined only to the TB varieties analyzed in the present study (data not shown). Out of 19 loci, one locus RM 330 was found to be duplicated only in NB varieties (except PR 106) and none of the TB varieties showed any such duplication for this locus. None of the 19 SSR loci could distinguish the TB from the EB varieties independently. However, a combination of the polymorphic loci with different Basmati specific alleles enabled discrimination of the traditional from the evolved ones except CSR 30B. For example, RM 171 locus in combination with RM 238 or RM 16 or RM 302 could discriminate all the traditional ones from the evolved ones (except CSR 30B) (Please refer Table 7 here below).

TABLE 7
SSR loci that distinguish pure Basmati and evolved Basmati rice varieties

| | | | | | |
|---|---|---|---|---|---|
| | | | Allele | Pure | |
| LOCUS | Forward primer | Reverse primer | Size (bp) | Basmati | Evolved Basmati varieties |

| | | | | | | |
|---|---|---|---|---|---|---|
| RM 163 | ATCCATGTGCGCCTTTATGAGGA | CGCTACCTCCTTCACTTACTAGT | 140 | − | + (except 385, SB, KAR) | |
| RM 171 | AACGCGAGGACACGTACTTAC | ACGAGATACGTACGCCTTTG | 445 | − | + (except 385, TER) | |
| ″ | ″ | ″ | 438 | + | − | − (except 385 CSR) |
| RM 72 | CCGGCGATAAAACAATGAG | GCATCGGTCCTAACTAAGGG | 175 | + | − (except 385, SB) | |
| RM 102 | AACTTTCCCACCACCACCGCGG | AGCAGCAGCAAGCCAGCAAGCG | 140 | + | − (except KAR, PB, SB) | |
| RM 238 A | GATGGAAAGCACGTGCACTA | ACAGGCAATCCGTAGACTCG | 445 | + | − (except PB, SB) | |
| RM 16 | CGCTAGGGCAGCATCTAAAA | AACACAGCAGGTACGCGC | 165 | + | − (except SB, HB) | |
| RM 302 | TCATGTCATCTACCATCACAC | ATGGAGAAGATGGAATACTTGC | 140 | + | − (except KAR, PB, SB) | |
| RM 330 A | CAATGAAGTGGATCTCGGAG | CATCAATCAGCGAAGGTCC | 180 | − | − | + (except 385, PB, SB, KAR) |
| RM 136 | GAGAGCTCAGCTGCTGCCTCTAGC | GAGGAGCGCCACGGTGTACGCC | 124 | − | + (except 385, KAR, SB) | |

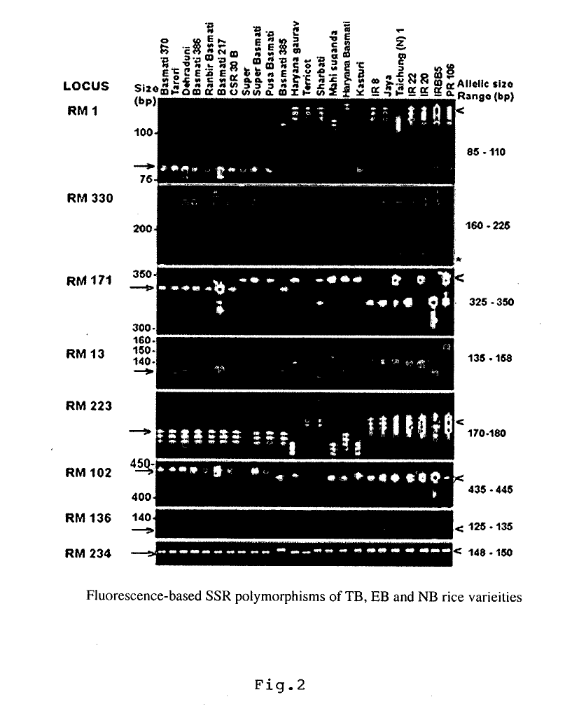

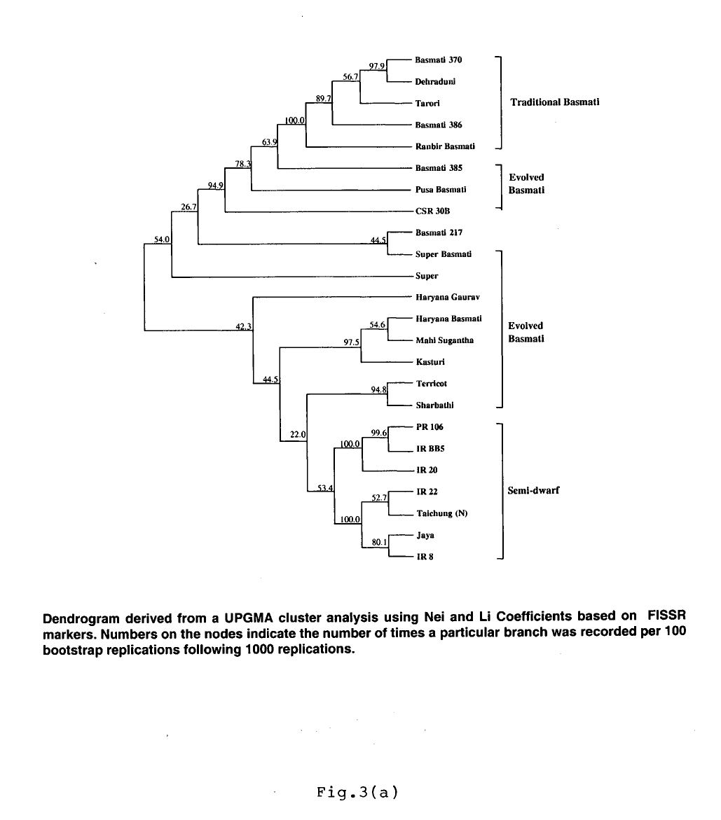

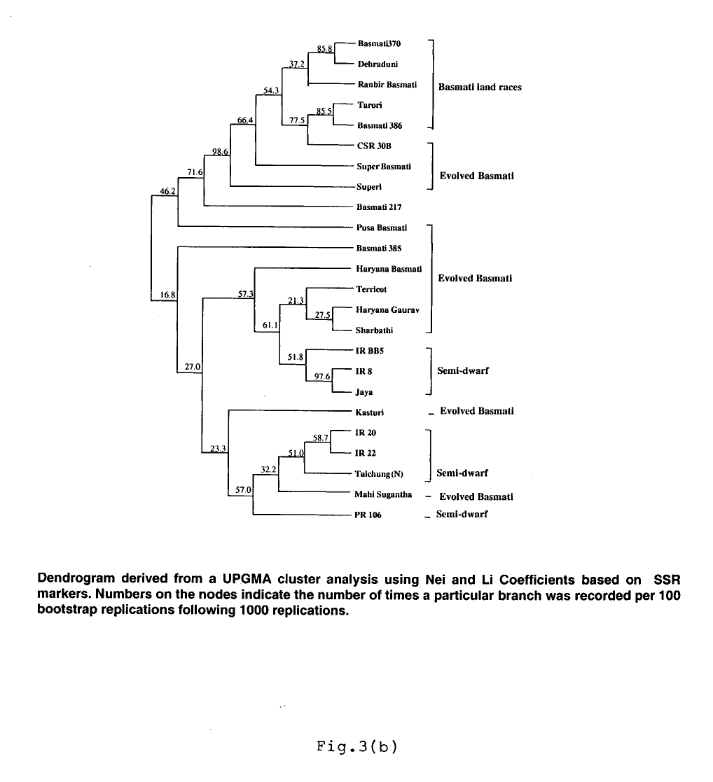

In still another embodiment of the present invention, wherein the ISSR-PCR and SSR profiles were used to determine the genetic similarity matrices, which were then used to construct dendrograms. Both the methods separated the TB from the NB varieties (FIG. 3). The EB varieties got clustered in between depending upon the degree of genetic similarity to the two groups. In ISSR-PCR analysis, all the TB varieties except Basmati 217, displayed 95% similarity among themselves. Genetic similarity estimates obtained by SSR analysis also revealed similar results. Both the marker assays included the land race Basmati 217 along with the EB varieties. As revealed by ISSR analysis, among the EB varieties, CSR, SB, 385, PB and SU showed higher genetic similarity to the TB varieties as compared to the other EB varieties which showed higher similarity to NB varieties (FIG. 2).

In still another embodiment of the present invention, wherein with SSR markers (FIG. 2) the evolved Basmati have clustered along with Non-Basmati cultivars. Thus, ISSR primers are better than SSR markers in separating Evolved Basmati from Non-Basmati.

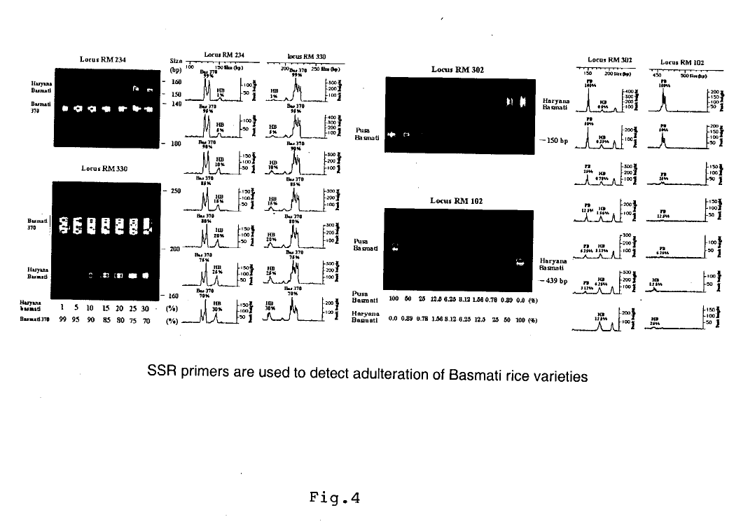

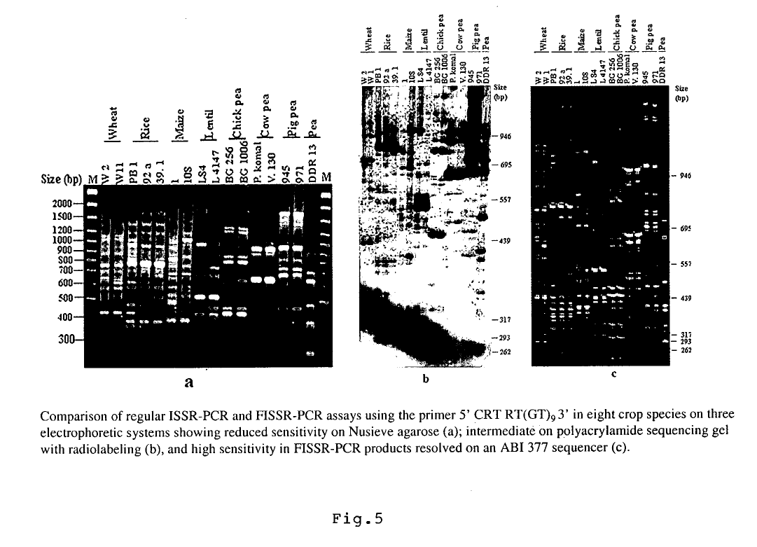

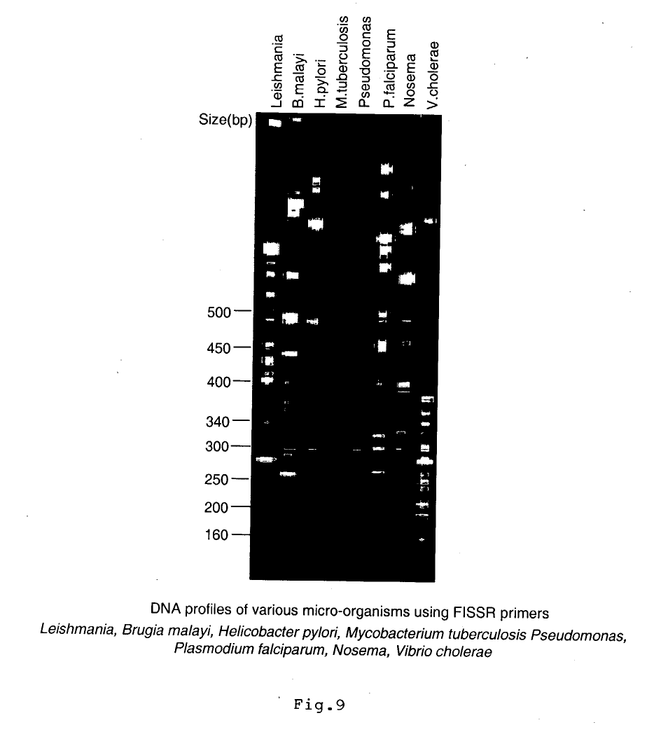

In still another embodiment of the present invention, wherein it is demonstrate that the sensitivity, speed and informativeness of the existing ISSR-PCR method could be enhanced substantially by using fluorescent dye labeled nucleotide in the ISSR-PCR reaction, which we call FISSR-PCR, followed by separation of PCR products on an ABI automated sequencer 377, using diverse species of plants, and parasitic organisms (FIG. 5 and FIG. 9).

In still another embodiment of the present invention, wherein Plant genomic DNA was extracted as described by Dellaporta et al.³⁸ Briefly, the seeds were ground in 200 μl of grinding/lysis buffer (50 mM Tris-HCl pH 7.2, 50 mM EDTA pH 8.0, 3% SDS, 1% β-mercaptoethanol). The DNA was precipitated with 0.1 final volume of 3.0 M sodium acetate pH 5.2 and 2.5 volume of absolute ethanol, incubated at −70° C. DNA was quantified on 0.8% agarose gel and diluted to a uniform concentration (10 ng/μl). Genomic DNA from silk moths was isolated either from the posterior silk glands collected from day 3 fifth instar larvae or from the whole pupae.¹⁶ Briefly, silk glands or whole pupae were ground in liquid nitrogen using a pestle and mortar. Extraction buffer (100 mM Tris-HCl, pH 8.0, 50 mM NaCl, 50 mM EDTA and 1% SDS) was added to the ground tissue and incubated at 37° C. for 2 hrs with occasional swirling. The DNA was extracted twice with phenol-choloroform-isoamyl alcohol (25:24:1) and once with chloroform. The supernatant DNA was ethanol precipitated, resuspended in TE (10 mM Tris-HCl, 1 mM EDTA, pH 8.0) buffer and incubated at 37° C. for 1 h after addition of RNase A (100 μg/ml).

In still another embodiment of the present invention, wherein we designed and synthesized in-house six 5′-anchored primers SEQ ID NO. 1, SEQ ID NO.3, SEQ ID NO. 4, SEQ ID NO. 5, SEQ ID NO. 6, and SEQ ID NO. 7 and two 3′-anchored primers of SEQ ID NO. 26 and SEQ ID NO. 27 on an ABI DNA synthesizer, purified by PAGE and then desalted through an oligonucleotide purification cartridge (OPC).

In still another embodiment of the present invention, wherein Eight different plant species of Wheat, Rice, Maize, Lentil, ChickPea, CowPea, Pigeon Pea, and Pea and two to three varieties from each of the plant species were used for FISSR analysis (FIG. 5).

In still another embodiment of the present invention, wherein for FISSR-PCR assay, amplification was performed in 10 mM Tris-HCl, pH 8.3/50 mM KCl/2.5 mM MgCl₂/0.01% gelatin/0.01% Triton X-100/1 mM dNTPs/0.4 μM Fluorescent dUTP (TAMARA Perkin Elmer)/0.25 unit of Taq DNA polymerase (Ampli Taq Gold, Perkin Elmer), 4 μM primer with 5 ng of genomic DNA per 5 μl reaction. The thermal cycling conditions were as follows: initial denaturation of 10 min at 94° C., 35 cycles of 30 s at 94° C., 30 s at 50° C. and 1 min at 72° C., final extension 10 min at 72° C. The PCR was performed on a DNA thermal cycler (Perkin Elmer 9600). One μl of PCR products was mixed with 1.5 μl of 6× loading buffer (1:4 mixture of loading buffer and formamide (Sigma) and 0.4 μl of Gene scan-1000 (ROX) size was included in the loading samples.

In still another embodiment of the present invention, wherein the samples were denatured at 92° C. for 1 min prior to loading on to an ABI 377 automated sequencer and electrophoresed on 5% polyacrylamide gel (Long ranger, FMC) under denaturing conditions containing 7 M urea, in 1×TBE buffer (90 mM Tris borate, pH 8.3 and 2 mM EDTA). We also analyzed ISSR-PCR products on agarose gel electrophoresis. (FIG. 5)

In still another embodiment of the present invention, wherein to compare the efficacy of the FISSR-PCR assay with the regular ISSR-PCR assay, the ISSR-PCR reactions were carried out. The PCR and thermal cycling conditions used were as in FISSR-PCR assay except 20 ng template DNA and 8 μM primer without fluorescent dUTP were used in 10 μl reaction. The PCR products were electrophoresed on 2% agarose (50% Sigma+50% Nusieve) for 3 hrs at 100V (FIG. 5). The ISSR-PCR reactions were also carried out as previously reported²⁸ in the presence of α[³²P] labeled dCTP and cold dCTP (added in the ration of 1:4) in the PCR reaction. The PCR products were denatured at 75° C. for 2 min, chilled on ice and electrophoresed on a standard sequencing gel (5% acrylamide, 7M urea, 1×TBE) and run at 900 V under constant power supply for 15 hrs (FIG. 5).

In still another embodiment of the present invention, wherein each of the 8 SSR-anchored primers revealed distinct PCR profiles in agarose, PAGE (PCR products incorporated with α[32P] labeled nucleotide) and ABI 377 automated sequencer (PCR products incorporated with fluorescent nucleotide) in all the 8 plant species with characteristic species and varietal specific profiles as shown in FIG. 5. However, the number of markers revealed in each of the methods differed substantially. The FISSR assay was found to be the most informative in all the species studied. It resolved almost two-fold more number of markers as compared to the existing ISSR-PCR methods based on agarose and PAGE analyses. As a result, the number of species and varietal specific markers available for scoring increased substantially (FIG. 3, and Table 8). For example, the primer SEQ ID NO. 5 in FISSR assay resolved 45 scorable bands in rice out of which as many as 14 markers were specific to rice as compared with the other seven plant species. Ten markers could distinguish all the 3 rice varieties.

In still another embodiment of the present invention, wherein since FISSR-PCR technique is very sensitive it is a method of choice for detecting polymorphic markers in closely related varieties/populations which are otherwise difficult to discriminate by using other marker systems. The inter-varietal polymorphisms among the self pollinated and most conserved legume varieties suggest the efficiency and usefulness of this assay for varietal and cultivar identification. The FISSR-PCR assay could be successfully used as a rapid, sensitive and informative technique to quickly fingerprint a large number of genotypes of a given species in a cost-effective manner. Since FISSR-PCR assay is automated, a single assay including analysis could be completed within a day.

In still another embodiment of the present invention, wherein the method is also very useful to quickly evaluate the abundance of microsatellite repeats in different genomes using anchored primers with different microsatellite motifs. For example, (GT)ₙ/(CA)ₙ is more abundant in wheat and rice genomes than other genomes studied. Such information is crucial in the projects aimed at microsatellite marker development³⁰

TABLE 8
Comparison of FISSR-PCR assay using SEQ ID NO. 5 with regular
ISSR−PCR based on agarose gel and PAGE analysis

| | | Total | Molecular |
|---|---|---|---|
| | | number of | size |
| Crop | Assay | bands | (bp) |
| Wheat | Agarose ISSR-PCR | 18 (06, 02) | 300-1900 |
| | PAGE ISSR-PCR | 22 (05, 03) | 290-1200 |
| | FISSR-PCR | 47 (14, 03) | 250-1200 |
| Rice | Agarose ISSR-PCR | 21 (06, 04) | 250-1700 |
| | PAGE ISSR-PCR | 24 (05, 03) | 260-1100 |
| | FISSR-PCR | 45 (14, 10) | 250-1200 |
| Maize | Agarose ISSR-PCR | 14 (08, 03) | 300-1000 |
| | PAGE ISSR-PCR | 21 (09, 02) | 270-1200 |
| | FISSR-PCR | 32 (12, 05) | 260-1100 |
| Lentil | Agarose ISSR-PCR | 13 (04, 01) | 300-1000 |
| | PAGE ISSR-PCR | 23 (07, 02) | 280-1200 |
| | FISSR-PCR | 32 (14, 02) | 250-1200 |
| Chick Pea | Agarose ISSR-PCR | 14 (08, 02) | 350-1200 |
| | PAGE ISSR-PCR | 16 (08, 03) | 250-1200 |
| | FISSR-PCR | 22 (10, 00) | 250-1200 |
| Cow Pea | Agarose ISSR-PCR | 08 (04, 02) | 350-0900 |
| | PAGE ISSR-PCR | 10 (05, 03) | 250-1200 |
| | FISSR-PCR | 19 (08, 03) | 250-1000 |
| Pigeon Pea | Agarose ISSR-PCR | 12 (04, 02) | 400-1700 |
| | PAGE ISSR-PCR | 17 (08, 02) | 260-1200 |
| | FISSR-PCR | 33 (16, 02) | 250-1200 |
| Pea | Agarose ISSR-PCR | 09 (05, —) | 270-0900 |
| | PAGE ISSR-PCR | 20 (12, —) | 250-1000 |
| | FISSR-PCR | 26 (08, —) | 290-1000 |

Figures in the parentheses indicate species and varietal specific markers, respectively.

In still another embodiment of the present invention, wherein we also used this technique successfully to amplify the diverse genomes of insects, parasites of insects and human such as Plasmodium, Leishmania and Brugia malayi and many infectious organisms such as Vibrio cholerae Mycobacterium tuberculosis, Helicobacterium pylori, Pseudomonas aeruginosa and other organisms (FIG. 9) where DNA yield per sample may be too low to be useful for conventional PCR-based marker assays. In all the cases, very clear, easily scorable and highly reproducible FISSR markers could be resolved depending on the type of core microsatellite repeat included in the anchored ISSR primers.

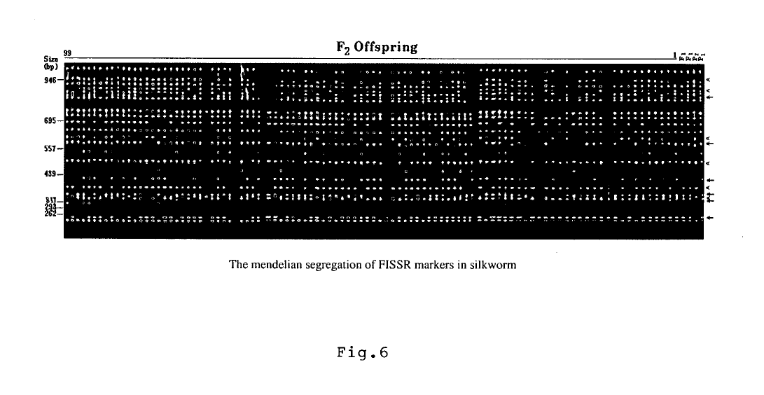

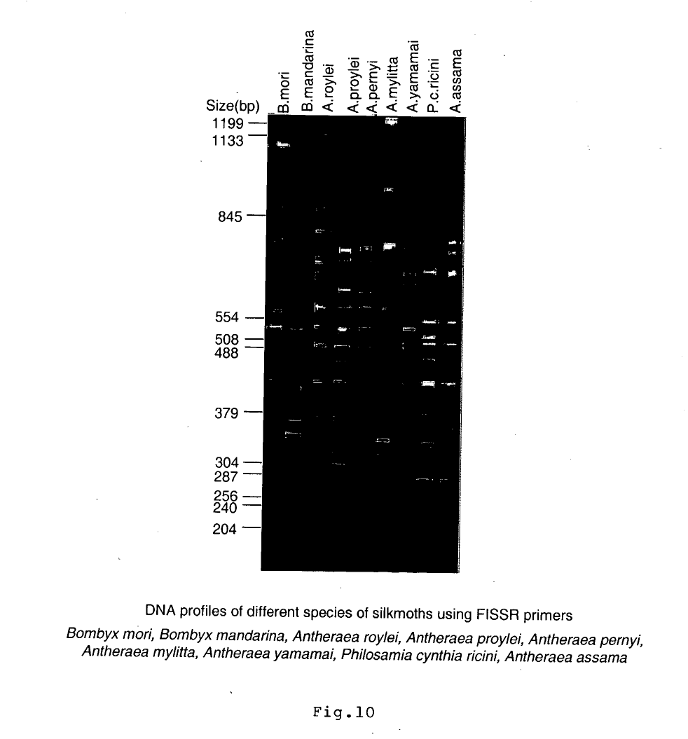

In still another embodiment of the present invention, wherein An F₂ mapping population of 99 offspring developed by sib-mating of F₁ hybrid silk moths derived from a cross of two divergent strains of B. mori, P⁵⁰×C₁₀₈ was used to demonstrate the inheritance and segregation of FISSR markers and thus showing FISSR-PCR is a robust method for high resolution mapping of complex genome (FIG. 6 and FIG. 10)

In still another embodiment of the present invention, wherein to demonstrate the utility of FISSR-PCR markers in genetic mapping experiments, we analyzed 99 F₂ offspring of silkworm, B. mori derived from a cross of two divergent silkworm strains, P⁵⁰ and C₁₀₈ using the markers amplified by the primer, SEQ ID NO. 7. As could be seen from FIG. 6 and Table 9, the 12 markers which were polymorphic between the two parental strains inherited and segregated according to Mendelian principle. These results show that the FISSR-PCR markers are very useful for quick and high throughput genotyping of mapping population using very small quantity of template DNA. For constructing high density linkage maps, a large number of F₂ individuals need to be analyzed using a number of molecular markers and the template DNA requirement in the case of other conventional PCR methods is relatively high. The FISSR assay provides a large number of DNA markers per primer and allows detection of markers with as little as 2-5 ng of template DNA, on an automated sequencer obviating the necessity for using radioactive isotopes.

TABLE 9
Mendelian segregation of FISSR-PCR markers amplified by RAY RAT
RC(GA)₇ in 99 F₂ offspring derived from P⁵⁰ and C₁₀₈ cross in silkworm.

| Polymorphic | | No. of F₂ | | | | |
|---|---|---|---|---|---|---|
| markers (bp) | | offspring | | Observed | | |
| P⁵⁰ | C₁₀₈ | scored | Expected Ratio | Ratio | χ² | P> |
| | 940 | 99 | 74.25:24.75 | 70:29 | 0.973 | 0.300 |
| | 850 | 99 | 74.25:24.75 | 70:29 | 0.973 | 0.300 |
| 835 | | 99 | 74.25:24.75 | 67:32 | 2.876 | 0.100 |
| | 575 | 99 | 74.25:24.75 | 69:30 | 1.485 | 0.200 |
| 560 | | 99 | 74.25:24.75 | 75:24 | 7.600 | 0.006 |
| | 500 | 99 | 74.25:24.75 | 70:29 | 0.973 | 0.300 |
| 420 | | 99 | 74.25:24.75 | 74:25 | 10.943 | 0.001 |
| | 390 | 99 | 74.25:24.75 | 78:21 | 0.379 | 0.500 |
| 340 | | 99 | 74.25:24.75 | 77:22 | 0.406 | 0.500 |
| | 335 | 99 | 74.25:24.75 | 72:27 | 0.372 | 0.500 |
| 330 | | 99 | 74.25:24.75 | 75:24 | 7.600 | 0.005 |
| 260 | | 99 | 74.25:24.75 | 73:26 | 0.084 | 0.800 |

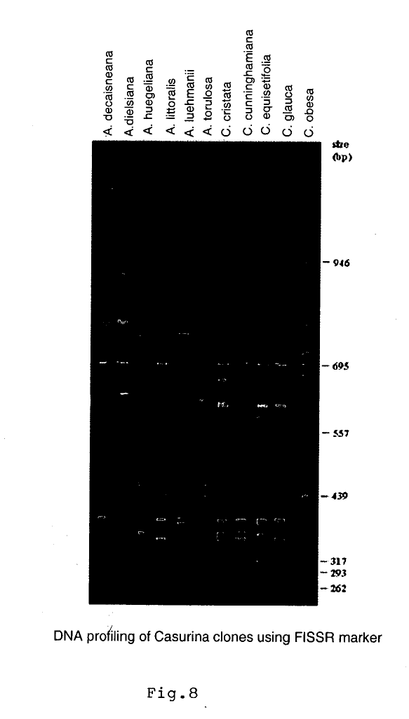

In still another embodiment of the present invention, wherein diverse Casuarina species were analyzed using FISSR PCR method. (FIG. 8)

In still another embodiment of the present invention, wherein in conclusion, we show that this rapid, less hazardous, simple and informative assay could be used in large scale screening of varieties/inbred lines, high resolution genetic mapping of complex genomes and quick genetic analysis of large sample sizes of various infectious organisms which yield very little quantity of DNA, with high degree of accuracy.

In still another embodiment of the present invention, wherein the traditional and evolved Basmati varieties included in the present study probably represent a major component of the Basmati gene pool of the Indian sub-continent. In addition to the Basmati varieties, we also included in our study many semi-dwarf non-Basmati varieties, some of which have been utilized for development of EB varieties. We used two molecular marker assays, fluorescent based ISSR-PCR and SSR of which the former is an improvised version of ISSR-PCR method developed earlier ¹⁵.

In still another embodiment of the present invention, wherein the number of bands produced across 24 rice varieties by different anchored SSR motifs is consistent with the published reports on microsatellite frequency in the rice genome. Among dinucleotide repeats, (GA)ₙ and (CA)ₙ are the most abundant in the rice genome^{9,12,14}. Both the repeat classes were amenable to fluorescent-based ISSR-PCR analysis of the rice genome as both were equally polymorphic. Out of the two 3′ anchored primers, one with (GA)ₙ and another with (GT)ₙ motif, the (GA)ₙ produced more number of bands probably due to its greater abundance in the rice genome as reported earlier^{9,14}. The 2 (GT)ₙ based primers, one anchored at the 5′ and the other at the 3′ end amplified 61 and 31 bands respectively. Although comparison of only two primers may not allow us to make definitive conclusions, taken together the earlier inferences¹⁶, such a difference may be due to the lack of selective nucleotides at the 3′ end of the 5′ anchored primers. On the other hand 5′ and 3′ anchored (GA)ₙ repeats did not reveal any such difference probably because 5′ anchored primers impose selection for long stretch of SSRs, while amplification with 3′ anchored primer would not impose selection for repeat length. Since (GA)ₙ motifs are reported to be longer in the rice genome as compared to (GT)ₙ motifs, both 5′ and 3′ anchored (GA)ₙ primers produce similar number of bands. On the other hand the lack of length advantage in (GT)ₙ motif probably results in difference between 5′ and 3′ end anchored (GT)ₙ primers.

In still another embodiment of the present invention, wherein all the (ATT)ₙ and (GACA)₄ anchored primers amplified a large number of bands in the present study in contrast to earlier studies¹⁶. Although (GACA)ₙ repeats are fewer than the di- and trinucleotide repeats they appear to be in good number in the rice genome as reported earlier³². Since the lengths of tri- and tetranucleotide repeats in the rice genome are mostly 5 to 8 and 5 to 6 respectively, the longer repeat motifs used by earlier studies¹⁶ would have precluded the amplification. Our results indicate that tri- and tetranucleotide bsed ISSR-PCR markers could provide potential markers in the rice genome.

In still another embodiment of the present invention, wherein there has been wide range of interests in the genetic differences between TB, EB and NB rice varieties. The information available on genetic diversity and differences of the three groups is scanty except for the Asian rice varieties using isozyme markers³. The results of the present study using fluorescent-based ISSR and SSR markers indicate that TB varieties have the least diversity as compared to the EB and NB varieties. Besides, both the marker assays showed that there are no significant differences between the TB varieties. We believe that the high degree of genetic similarity among the TB varieties indicate that they are possibly the descendents of a single land race and the minor genetic variation is maintained as a result of selection and preference imposed by farmers for several years. This observation draws support from the historical relationship of Basmati varieties used in the present study. Most of the TB varieties classified under different names are likely to have been selected from the local variety such as Basmati 370 released for commercial cultivation in 1933 at the Rice Research Station, Kalashah Kaku (now in Pakistan). For example, the isozyme patterns of 60 out of 65 Pakistani accessions described as Basmati matched the isozyme pattern of Basmati 370 and Type 3. Similarly, of the nine varieties from India all except Karnal local were identical to the isozyme pattern of Basmati 370 and Type 3 (36). Among the 19 (33) and 5 (34) EB varieties released since 1965 for cultivation in India and Pakistan respectively, 12 and 4 had Basmati 370 as one of the donor parents. Our studies along with these reports suggest that the TB varieties used in the present study could be considered as bulk of the narrow TB gene pool of the Indian sub-continent. Recent report on RAPD profiling of aromatic rices also shows low level of genetic diversity (35). The variety, Basmati 217 which we received as land race showed only 75% and 66% similarities whereas, CSR 30B which was received as EB showed 82% and 96% similarities based on ISSR and SSR marker assays respectively, to the five TB varieties. Considering the genetic diversity in the other EB varieties it is unlikely that CSR 30B evolved from a direct cross between a Pakistani Basmati variety and the salt tolerant non-Basmati variety, Buraratha, has attained such high level of genetic similarity to the TB varieties. However, further insights into its grain quality vis-a vis TB and EB varieties and the pedigree details may be required for ascertaining its status. A reasonable explanation for the higher genetic distance of Basmati 270 from the other TB varieties is that it has differentiated from the rest of the TB varieties and probably represents a separate lineage. This is supported by the observation that out of the 70 SSR alleles among 24 varieties, 3 were unique to Basmati 217. The high level of genetic diversity and preponderance of NB alleles in most of the EB varieties indicate that most of them still retain a large genomic fraction of the NB varieties used in the breeding program.

In still another embodiment of the present invention, wherein both the marker assays clearly differentiate the Basmati and non-Basmati varieties as highlighted by isozyme and RFLP studies³. The high level of genetic differentiation of Basmati and NB rice varieties suggests that the former might have possibly diverged a long time ago from NB varieties through conscious selection and patronage. The duplication event at locus RM 330 only in semi-dwarf non-Basmati varieties including the old japonica varieties, Taipai and Wu 10B supports the divergence of aromatic varieties from the common ancestor. It would be interesting to study the other varieties in Group V, which embraces most of the long grain varietes (3), for duplication event in this locus. The high level genetic differentiation of the two groups may be the possible reason for lack of finding desirable recombinants and introgression of useful genes in Basmati breeding programmes (2). This is reflected by the preponderance of NB alleles in most of the EB varieties. Some of the bands/alleles unique to the TB varieties used in the present study are not at all found in any of the EB varieties either because of incompatible chromosome regions (coadapted gene complex) or they are possibly linked to the negative traits of Basmati and are thus selected against. On the other hand, alleles from the NB varieties have survived in greater number in the EB varieties possibly because they are in close proximity to the genes, which confer useful traits of NB varieties.

In still another embodiment of the present invention, wherein the high resolution of fluorescence based ISSR-PCR assay described in the present study provides a large number of highly reproducible markers using as little as 5 ng template DNA per PCR reaction without using radioactive isotopes or chemicals involved in silver staining. By varying SSR motifs and their anchors a large number of markers could be generated which can be used for further saturation of the rice genome. We believe that since bulk of the global Basmati rice trade and breeding programs center around the TB varieties analyzed in the present study, the combination of SSR and ISSR markers can be used to identify these varieties from both NB as well as EB varieties. This will also enable the determination of adulteration of this set of traditional Basmati varieties. It is hazardous to venture to the conclusion that the markers observed in the present study could be universally applied to all aromatic rice varieties. Further studies on geographically random samples of aromatic rice varieties would probably give a more complete and less discontinuous picture with regard to the allelic/marker association. Nevertheless, the markers specific to the TB varieties used in the present study should be further pursued to look for allelic association, if any, with the Basmati phenotype. Such a study would provide markers which would help to eliminate unnecessary chromosome regions in the early stages of backcrossing, thus helping the breeders to shorten the breeding cycles by rapid incorporation of Basmati traits into breeding lines.

In still another embodiment of the present invention, wherein recent progress in DNA marker technology, particularly PCR based markers have augmented the molecular marker resources for the genetic analysis of a wide variety of genomes. As PCR technology finds increased use in various genetic analyses, additional novel variations of this technique are emerging. PCR analysis using anchored simple sequence repeat primers has gained attention recently as an attractive means of characterizing genomes. In the present invention we have developed a technique which we have termed as FISSR-PCR (for fluorescent-inter-simple-sequence-PCR) which makes use of the fluorescent dye labeled nucleotide in the PCR reaction, followed by separation of PCR products on an ABI automated sequencer 377, using template DMA from diverse species of plants and their varieties and animals. We show that the FISSR-PCR polymorphic markers could be unambiguously scored to provide varietal and species specific molecular profiles. Besides, the FISSR-PCR markers could be followed in the segregating population such as second filial generation (F₂) thus showing their applicability in high resolution mapping of complex genomes.

In still another embodiment of the present invention, wherein the studies using anchored primer based PCR have shown that the polymorphisms could be detected in a variety of complex genomes. The applicant has extended the study to address the issue of automating the method by using fluorescent oligonucleotides in the PCR reaction and a variety of combinations of 5′ and 3′ anchored microsatellite primers and resolution of the PCR products on an automated sequencer. This would increase the sensitivity and speed of the assay by several manifolds thus providing an easy handle to the geneticists for high-resolution maping of complex genomes and identification and authentication of varieties and species using as little as 2 to 5 ng DNA per assay.

In still another embodiment of the present invention, wherein we assayed eight plant species (2 varieties from each plant species except rice and pea which involved three and one sample respectively eg. Casuarina), using as many as 36 anchored primers designed by us. We genotyped 100 offspring of second filial hybrid offspring along with their first filial hybrid offspring and their parental strains of silkworm. In all the cases DNA was extracted from liquid nitrogen frozen tissue samples stored at −70° C. High molecular weight genomic DNA was extracted from all the plant and animal samples, quantified by spectrophotometer as well as by agarose gel electrophoresis and used as PCR templates. Each of the different 5′ and 3′ anchored microsatellite primers (Table 1) was tested on each of the template DNA samples.

In still another embodiment of the present invention, wherein the PCR reactions were carried out in a 5 microliter reaction volume carrying 5 ng of genomic DNA, 25 μM each of dCTP, dGTP, dTT and dATP, 0.8 μM anchored primer, and 0.4 μM fluorescent dUTP (Tamara dye, Perkin Elmer) using Taq gold DNA polymerase (Perkin Elmer). Amplification was performed on a termal cycler (Model 2400, from Perkin Elmer) with a programme of initial denaturation at 94° C. for 10 minutes followed by 35 cycles of 94° C. for 1 minute, 50° C. for 1 minute and 72° C. for 2 minutes followed by final extension at 72° C. for 10 minutes, and finally stored at 4° C.

In still another embodiment of the present invention, wherein the products were run on 5% polyacrylamide gel with 7M urea, on an ABI automated sequencer 377 at a constant voltage of 3000 volts for 7 hours. The data were analysed using Genescan analysis software. Under optimized reaction conditions, the replicate experiments were carried out and only consistently reproducible bands were scored for genotyping varieties, species and clones.

In another main embodiment of the present invention, wherein SSR markers are used to detect the adulteration of Basmati rice varieties (FIG. 4). For example, basmati 370, a traditional popular Basmati (TB) variety is adultered with an Evolved Basmati (EB) variety, Haryana Basmati at different levels i.e., 70:30 (TB:EB), 75:25 (TB:EB), 80:20 (TB:EB), 85:15 (TB:EB), 90:10 (TB:EB), 95:5 (TB:EB), 99:1 (TB:EB). The DNA extracted for the adultered sample was analyzed using RM 234 and RM 330 primers in independent experiments. The peaks generated scanning the intensity of the alleles, reveal that adulteration of Traditional Basmati by even 1% of Evolved Basmati could be detected in a sample.

In another main embodiment of the present invention, wherein DNA profiles of different species of silk moths is shown (FIG. 10) using FISSR primers. The silk moths used for profiling comprises Bombyx mori, Bombyx mandarina, Antheraea roylei, Antheraea proylei, Antheraea pernyl, Antheraea mylitta, Antheraea yamamai, Philosamia cynthia ricini, and Antheraea assama.

In still another embodiment of the present invention, wherein said experiment (FIG. 10) further establishes that the FISSR-PCR primers can be used for genotyping eukaryotes including silk moths.

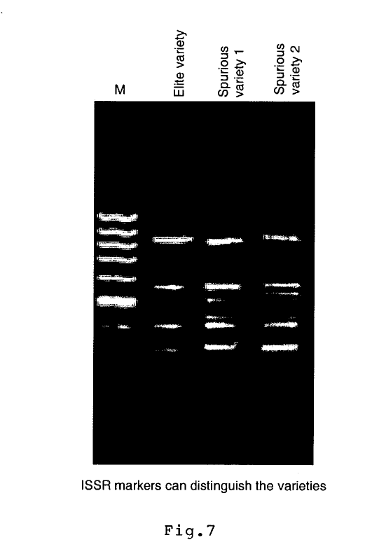

In yet another embodiment of the present invention, wherein the spurious varieties are sorted out from the elite verities (FIG. 7) using ISSR markers.

### REFERENCES

1. Singh, V. P (2000) in Aromatic Rices, eds. Singh, R. K., Singh, U. S. & Khush, G. S. (Oxford & IBH, New Delhi), pp135-153.

2. Khush, G. S., Paule, C. M. & De La Cruz, N. M. (1979) Proc. Workshop on Chemical aspects of rice grain quality. Int. Rice Res. Inst., Los Banos, Institute, Manila, Philippines. Laguna, Philippines, pp 21-31.

3. Glaszmann, J. C. (1987) Theor. Appl. Genet. 74, 21-30.

4. Engle, L. M., Chang, T. T. & Ramirez, D. A. (1969). Philipp. Agric. 53, 289-307.

5. Botstein, D. R., White, R. L., Skolnick, M. & Davis, R. W. (1980). Am. J. Hum. Genet. 32, 314-331.

6. Williams, J. G. K., Kubelik, A. R., Livak, K. J., Rafalski, J. A. & Tingy, S. V. (1990) Nucleic Acids Res. 18, 6531-6535.

7. Weber, J. L. & May, P. E. (1989) Am. J. Hum. Genet. 44, 388-396.

8. Vos, P., Hogers, R., Bleekr, M., Reijans, M. & Zabeau, M. (1995) Nucleic Acids Res. 23, 4407-4414.

9. Wu, K. & Tanksley, S. D. (1993) Mol. Gen. Genet. 241, 225-235.

10. Panaud, O., Chen, X. & McCouch, S. R. (1995) Genome 38, 1170-1176.

11. Yang, G. P., Saghai Maroof, M. A., Xu, C. G., Zhang, Q. & Biyashev, R. M. (1994) Mol. Gen. Genet. 245, 187-194.

12. Cho, Y. G., Ishii, T., Temnykh, S., Chen, X., Lipovich, L., McCouch, S. R., Park, W. D., Ayres, N. & Cartinhour, S. (2000) Theor. Appl. Genet. 100, 713-722.

13. Akagi, H., Yokozeki, Y., Inagaki, A. & Fujimura, T. (1996) Theor. Appl. Genet. 93, 1071-1077.

14. Temnykh, S., Park, W. D., Ayres, N., Cartinhour, S., Hauck, N., Lipovich, L., Cho, Y. G., Ishii, T. & McCouch, S. R. (2000) Theor. Appl. Genet. 100, 698-712.

15. Zietkiewicz, E., Rafalski, A. & Labuda, D. (1994) Genomics 20, 176-183.

16. Blair, M. W., Panaud, O. & McCouch, S. R. (1999) Theor. Appl. Genet. 98, 780-792.

17. Williams, J. G. K., Kubelik, A. R., Livak, K. J., Rafalski, J. A. & Tingey, S. V. (1990). DNA polymorphisms amplified by arbitrary primers are useful as genetic markers. Nucleic Acids Research 18, 6531-5.

18. Welsh, J. & McClelland, M. (1990). Fingerprinting genomes using PCR with arbitrary primers. Nucleic Acids Research 18, 7213-8. Vos, P., Hogers, R.,

19. Bleeker, M., Reijans, M., van de Lee, T., Homes, M., Frijters, A., Pot, J., Peleman J., Kuiper, M. & Zabeau, M. (1995). AFLP: a new technique for DNA fingerprinting. Nucleic Acids Research 23, 4407-14.

20. Tautz, D. (1989). Hypervariability of simple sequences as a general source for polymorphic DNA markers. Nucleic Acids Research 17, 6463-71.

21. Weber, J. L. & May, P. E. (1989). Abundant class of human DNA polymorphisms which can be typed using the polymerase chain reaction. American Journal of Human Genetics 44, 388-96.

22. Broun, P. & Tanksley, S. D. (1996). Characterization and genetic mapping of simple repeat sequences in the tomato genome. Molecular and General Genetics 250, 39-49.

23. Godwin, I. D., Aitken, E. A. & Smith, L. W. (1997). Application of inter simple sequence repeat (ISSR) markers to plant genetics. Electrophoresis 18, 1524-28.

24. Provan, J., Powell, W. & Waugh, R. (1996). Analysis of cultivated potato (Solanum tuberosum) using inter-microsatellite amplification. Genome 39, 767-9.

25. Tsumura, Y., Ohba, K. & Strauss, S. H. (1996). Diversity and inheritance of inter-simple sequence repeat polymorphisms in Douglas-fir (Pseudotsuga menziesii) and sugi (Cryptomeria japonica). Theoretical and Applied Genetics 92, 40-5.

26. Blair, M. W., Panaud, O. & McCouch, S. R. (1999). Inter-simple sequence repeat (ISSR) amplification for analysis of microsatellite motif frequency and fingerprinting in rice (Oryza sativa L.). Theoretical and Applied Genetics 98, 780-92.

27. Ratnaparkhe, M. B., Tekeoglu, M. & Muehlbauer, F. J. (1998). Inter-simple-sequence-repeat (ISSR) polymorphisms are useful for finding markers associated with disease resistance gene clusters. Theoretical and Applied Genetics. 97, 515-9.

28. Reddy, K. D., Nagaraju, J. & Abraham, E. G. (1999). Genetic characterization of the silkworm Bombyx mori by simple sequence repeat (SSR)-anchored PCR. Heredity 83, 681-7.

29. Nagaraju, J., Reddy, K. D., Nagaraja, G. M. & Sethuraman, B. N. (2001). Comparison of multilocus RFLPs and PCR-based marker systems for genetic analysis of the silkworm, Bombyx mori. Heredity 86, 588-97.

30. Panaud, O., Chen, X. & McCouch, S. R. (1996). Development of microsatellite markers and characterization of simple sequence length polymorphism (SSLP) in rice (Oryza sativa L.). Molecular and General Genetics 252, 597-607.

31. Dellaporta, S, L., Wood, J. & Hicks, J. B. (1983). A plant DNA preparation version II. Plant Molecular Biology Reporter 4, 19-21.

1. A set of inter-simple sequence repeats (ISSR)-PCR primers of SEQ ID Nos. 1 to 37 for genotyping eukaryotes.

2. A set of primers as claimed in claim 1, wherein primers of SEQ ID No. 1 to 25 are 5′ anchored primers.

3. A set of primers as claimed in claim 1, wherein primers of SEQ ID Nos. 26 to and 37 are 3′ anchored primers.

4. A method of genotyping diverse genomes of plant and animal systems using FISSR-PCR primers of claim 1, said method comprising steps of:
(a). extracting DNA from said systems,
(b). conducting a polymerase chain reaction (PCR) using extracted DNA, the said primers, and a flourescent label,
(c). obtaining a plurality of flourescent amplified products,
(d). separating the amplified products to produce fingerprint pattern using conventional techniques,
(e). identifying Monomorphism (M), and Polymorphism (P) amplified products, and
(f). genotyping the genomes of the said systems based on the polymorphic amplified fragments.

5. A method as claimed in claim 4, wherein the flourescent label can be selected from a group comprising Tamara dye, R6G and R110.

6. A FISSR method of distinguishing Basmati rice varieties from Non-Basmati (NB) rice varieties, using primers of SEQ ID Nos. 1-5, 7, 11, 19, 20, 25, 26, and 27, said method comprising steps of:
(a). extracting DNA from said rice varieties,
(b). conducting a polymerase chain reaction (PCR) using extracted DNA, the said primers, and a flourescent label,
(c). obtaining a plurality of flourescent amplified products,
(d). separating the amplified products to produce fingerprint pattern using conventional techniques,
(e). identifying Monomorphism (M), and Polymorphism (P) amplified products, and
(f). distinguishing Basmati rice varieties from Non-Basmati (NB) rice varieties based on the polymorphic amplified fragments

7. A method as claimed in claim 6, wherein the flourescent label can be selected from a group comprising Tamara dye, R6G and R110.

8. A FISSR method of distinguishing Traditional Basmati (TB) rice varieties from Evolved Basmati (NB) rice varieties, using primers of SEQ ID Nos. 1-5, 7, 11, 19, 20, 25, 26, and 27, said method comprising steps of:
(a). extracting DNA from said rice varieties,
(b). conducting a polymerase chain reaction (PCR) using extracted DNA, the said primers, and a flourescent label,
(c). obtaining a plurality of flourescent amplified products,
(d). separating the amplified products to produce fingerprint pattern using conventional techniques,
(e). identifying Monomorphism (M), and Polymorphism (P) amplified products, and
(f). distinguishing Traditional Basmati (TB) rice varieties from Evolved Basmati (EB) rice varieties based on the polymorphic amplified fragments

9. A method as claimed in claim 8, wherein the flourescent label can be selected from a group comprising Tamara dye, R6G and R110.

10. A method as claimed in claim 9, wherein the average number of bands produced by the primers with different repeat motifs negatively correlated with the number of nucleotides in the repeat unit of the motif.

11. A method as claimed in claim 8, wherein the number of products amplified in different repeat length classes reflect the frequency of different repeat motifs distributed in the rice genome.

12. A method of genotyping diverse genomes of plant and animal systems using SSR-PCR markers of table 3, said method comprising steps of:
(a). extracting DNA from said systems,
(b). conducting a polymerase chain reaction (PCR) using extracted DNA, the said primers, and a flourescent label,
(c). obtaining a plurality of flourescent amplified products,
(d). separating the amplified products to produce fingerprint pattern using conventional techniques,
(e). identifying Monomorphism (M), and Polymorphism (P) amplified products, and
(f). genotyping the genomes of the said systems based on the polymorphic amplified fragments.

13. A method as claimed in claim 12, wherein the flourescent label can be selected from a group comprising Tamara dye, R6G and R110.

14. A SSR method of distinguishing Basmati rice varieties from Non-Basmati (NB) rice varieties, using markers of table 4, said method comprising steps of:
(a). extracting DNA from said rice varieties,
(b). conducting a polymerase chain reaction (PCR) using extracted DNA, the said primers, and a flourescent label,
(c). obtaining a plurality of amplified products,
(d). separating the amplified products to produce fingerprint pattern using conventional techniques,
(e). identifying Monomorphism (M), and Polymorphism (P) amplified products, and
(f). distinguishing Basmati rice varieties from Non-Basmati (NB) rice varieties based on the polymorphic amplified fragments

15. A method as claimed in claim 6, wherein the flourescent label can be selected from a group comprising Tamara dye, R6G and R110.

16. A SSR method of distinguishing Traditional Basmati (TB) rice varieties from Evolved Basmati (NB) rice varieties, using markers of Table 5, said method comprising steps of:
(a). extracting DNA from said rice varieties,
(b). conducting a polymerase chain reaction (PCR) using extracted DNA, the said primers, and a flourescent label,
(c). obtaining a plurality of flourescent amplified products,
(d). separating the amplified products to produce fingerprint pattern using conventional techniques,
(e). identifying Monomorphism (M), and Polymorphism (P) amplified products, and
(f). distinguishing Traditional Basmati (TB) rice varieties from Evolved Basmati (EB) rice varieties based on the polymorphic amplified fragments

17. A method as claimed in claim 8, wherein the flourescent label can be selected from a group comprising Tamara dye, R6G and R110.

18. A kit for determining adulteration of Basmati rice with other rice varieties, said kit comprising
(a). at least one ISSR-PCR primers from a set of primers of SEQ ID Nos. 1 to 37, and/or
(b). at least one SSR markers from a set of markers of Table-4.

19. A method for determining adulteration of Basmati rice with other rice varieties using at least one ISSR-PCR primers from a set of primers of SEQ ID Nos. 1 to 37, and/or at least one SSR markers from a set of markers of Table-4, said method comprising steps of:
(a). extracting DNA from various rice varieties,
(b). conducting a polymerase chain reaction (PCR) using extracted DNA, the said primer(s) or marker(s), and a flourescent label,
(c). obtaining a plurality of flourescent amplified products,
(d). separating the amplified products to produce fingerprint pattern using conventional techniques,
(e). identifying Monomorphism (M), and Polymorphism (P) amplified products, and
(f). determining adulteration in Basmati rice varieties with other rice varieties based on the polymorphic amplified fragments.
